(19) United States
(12) Patent Application Publication
(10) Pub. No.: US 2006/0265049 A1
(43) Pub. Date: Nov. 23, 2006

(54) Stent and MR imaging process and device

(76) Inventors: Robert W. Gray, Rochester, NY (US); Howard J. Greenwald, Rochester, NY (US)

Correspondence Address: Robert W. Gray, 92 Cypress Street, East Rochester, NY, 14620, US

(21) Appl. No.: 11/132,676

(22) Filed: May 19, 2005

(51) Int. Cl. A61F 2/88

(52) U.S. Cl. 623/1.16; 600/420; 623/1.44

(57) ABSTRACT

A passive resonant circuit is disposed on an implanted stent. The materials, geomentry and electrical parameters of the stent with passive resonant circuit are chosen and arranged so that incident electromagnetic radiation induces currents in the passive resonant circuit that optimize imageability during MR scanning.

### FIELD OF THE INVENTION

A stent and an MRI process for the imaging the interior of a stent after it has been introduced into an object to be examined.

### BACKGROUND OF THE INVENTION

U.S. Pat. No. 6,280,385 of Andreas Melzer discloses and claims a novel stent assembly. Claim 10 of this patent, which is representative, describes “A stent imageable by a magnetic resonance imaging system and having a skeleton which can be unfolded, the stent comprising at least one passive resonance circuit having an inductor and a capacitor forming a closed-loop coil arrangement and whose resonance frequency corresponds to a resonance frequency of high-frequency radiation applied by the magnetic resonance imaging system.”

Although the stent disclosed in U.S. Pat. No. 6,280,385 has met with a reasonable degree of acceptance, it often does not have suitably low thrombogenic properties and a corresponding low potential for triggering an immune response when it is disposed within a biological organism. It is an object of this invention to provide a stent that has all of the desired properties of the stent of U.S. Pat. No. 6,280,385 but, in addition, has improved biocompatibility properties.

For a description of resonant circuits reference may be had, e.g., to Chapter 19, beginning at page 675, of J. Richard Johnson's “Electric Circuits” (Hayden Book Company, Hasbrouck Heights, N.J., 1984). Reference may also be had to TheFreeDictionary.com by Farlex which may be found at the Internet web site www.encyclopedia.thefreedictionary.com/RLC%20circuit and which states:

“In an electrical circuit, resonance occurs at a particular frequency when the inductive reactance and the capacitive reactance are of equal magnitude, causing electrical energy to oscillate between the magnetic field of the inductor and the electric field of the capacitor.

“Resonance occurs because the collapsing magnetic field of the inductor generates an electric current in its windings that charges the capacitor and the discharging capacitor provides an electric current that builds the magnetic field in the inductor, and the process is repeated. An analogy is a mechanical pendulum.

“At resonance, the series impedance of the two elements is at a minimum and the parallel impedance is a maximum. Resonance is used for tuning and filtering, because resonance occurs at a particular frequency for given values of inductance and capacitance. Resonance can be detrimental to the operation of communications circuits by causing unwanted sustained and transient oscillations that may cause noise, signal distortion, and damage to circuit elements.

“Since the inductive reactance and the capacitive reactance are of equal magnitude, ωL=1/ωC, where ω=2πf, in which f is the resonant frequency in hertz, L is the inductance in henries, and C is the capacitance in farads when standard SI units are used.”

TheFreeDictionary.com goes on to state: “The Q factor or quality factor is a measure of the “quality” of a resonant system. Resonant systems respond to frequencies close to the natural frequency much more strongly than they respond to other frequencies.

“On a graph of response versus frequency, the bandwidth is defined as the part of the frequency response that lies within 3 dB about the center frequency. . . .

“The Q factor is defined as the resonant frequency (center frequency f₀) divided by the bandwidth Δf or BW:

$$Q = \frac{{f}_{0}}{{f}_{2} - {f}_{1}} - \frac{{f}_{0}}{Δ f}$$

Bandwidth BW or Δf=f₂−f₁, where f₂ is the upper and f₁ the lower cutoff frequency. In a tuned radio frequency receiver (TRF) the Q factor is:

$$Q = \frac{1}{R} \sqrt{\frac{L}{C}}$$

where R, L, and C are the resistance, and capacitance of the tuned circuit, respectively.”

### SUMMARY OF THE INVENTION

In accordance with this invention, there is provided a stent assembly comprised of a stent, a first insulating material, a second insulating material, and a passive resonance circuit having an inductor, a resistor and a capacitor, wherein (a) the first insulating material is disposed on the stent, (b) the second insulating material is disposed on the inductor, (c) the stent is imageable by a magnetic resonance imaging system, and (d) the resonance frequency of the stent is with one kilohertz above or below the operating frequency of the magnetic resonance imaging system. Also in accordance with this invention, there is provided a stent assembly comprised of a stent, a passive resonance circuit having an inductor, a capacitor and a resistor wherein the stent assembly is imageable by a magnetic resonance imaging system.

### BRIEF DESCRIPTION OF THE DRAWINGS

Applicant's invention will be described by reference to this specification and to the enclosed drawings, in which like numerals refer to like elements, and wherein:

FIG. 1 is a schematic sectional view of one preferred stent assembly;

FIG. 2 is a stent with wire coil and coating capacitor on a staging area;

FIG. 2A is an expanded sectional view of a portion of the stent depicted in FIG. 2;

FIG. 3 is a stent with wire coil and coating capacitor;

FIG. 4 is a schematic diagram of a formation of a capacitor on a stent;

FIG. 5 is a schematic drawing of various inductor coil designs on a stent;

FIG. 6 is a schematic diagram of a formation of a capacitor on a stent strut;

FIG. 7 is a schematic diagram of a formation of a capacitor on a stent strut;

FIG. 8 is a graph of current versus frequency;

FIG. 9 is a graph of current versus frequency;

FIG. 10 is a graph of current versus frequency;

FIG. 11 is a stent with wire coil and coating capacitor at each end of the assembly;

FIG. 12 shows an experimental MRI image of stents;

FIG. 13 is a schematic diagram of an apparatus for determining resonance frequency;

FIG. 14 is a schematic diagram of an apparatus for determining resonance frequency;

FIG. 15 is a schematic diagram of a formation of a capacitor;

FIG. 16 is a schematic diagram of a formation of a capacitor; and

FIG. 17 is a schematic diagram of a formation of a capacitor.

### DESCRIPTION OF THE PREFERRED EMBODIMENT

The stent disclosed in this specification is an improvement upon the stents disclosed in U.S. Pat. No. 6,280,385, the entire disclosure of which is hereby incorporated by reference into this specification. Applicant's stent may incorporate one or more features of these prior art stents.

Claim 10 of U.S. Pat. No. 6,280,385 describes “A stent imageable by a magnetic resonance imaging system and having a skeleton which can be unfolded, the stent comprising at least one passive resonance circuit having an inductor and a capacitor forming a closed-loop coil arrangement and whose resonance frequency corresponds to a resonance frequency of high-frequency radiation applied by the magnetic resonance imaging system.” The entire disclosure of U.S. Pat. No. 6,280,385, as it relates to the stent described by such claim 10, is hereby incorporated by reference into this specification.

Claim 11 of U.S. Pat. No. 6,280,385 describes “11. The stent according to claim 10, wherein the skeleton of the stent acts as the inductor.” The entire disclosure of U.S. Pat. No. 6,280,385, as it relates to the stent described by such claim 11, is hereby incorporated by reference into this specification.

Claim 12 of U.S. Pat. No. 6,280,385 describes “12. The stent according to claim 11, wherein the skeleton is comprised of a material having at least one layer which is highly conductive.” The entire disclosure of U.S. Pat. No. 6,280,385, as it relates to the stent described by such claim 12, is hereby incorporated by reference into this specification.

Claim 13 of U.S. Pat. No. 6,280,385 describes “13. The stent according to claim 12, wherein the stent material comprises at least two layers, at least one layer having high conductivity and at least one layer having low conductivity.” The entire disclosure of U.S. Pat. No. 6,280,385, as it relates to the stent described by such claim 13, is hereby incorporated by reference into this specification.

Claim 14 of U.S. Pat. No. 6,280,385 describes “14. The stent according to claim 13, wherein the layer having high conductivity is separated at plural locations to define plural mutually insulated areas of the skeleton so as to form an inductor.” The entire disclosure of U.S. Pat. No. 6,280,385, as it relates to the stent described by such claim 14, is hereby incorporated by reference into this specification.

Claim 15 of U.S. Pat. No. 6,280,385 describes “15. The stent according to claim 13, wherein the skeleton comprises a honey-comb structure which is separated regularly above and beneath crossing points thereof.” The entire disclosure of U.S. Pat. No. 6,280,385, as it relates to the stent described by such claim 15, is hereby incorporated by reference into this specification.

Claim 16 of U.S. Pat. No. 6,280,385 describes “16. The stent according to claim 15, wherein the skeleton of the stent is configured as one of a helix, a double helix and multiple helixes.” The entire disclosure of U.S. Pat. No. 6,280,385, as it relates to the stent described by such claim 16, is hereby incorporated by reference into this specification.

Claim 17 of U.S. Pat. No. 6,280,385 describes “17. The stent according to claim 12, wherein the layer having high conductivity is separated at plural locations to define plural mutually insulated areas of the skeleton so as to form an inductor.” The entire disclosure of U.S. Pat. No. 6,280,385, as it relates to the stent described by such claim 17, is hereby incorporated by reference into this specification.

Claim 18 of U.S. Pat. No. 6,280,385 describes “18. The stent according to claim 10, wherein the inductor of the passive resonance circuit comprises a separate coil which is integrated into the stent.” The entire disclosure of U.S. Pat. No. 6,280,385, as it relates to the stent described by such claim 18, is hereby incorporated by reference into this specification.

Claim 19 of U.S. Pat. No. 6,280,385 describes “19. The stent according to claim 18, wherein the coil is woven into the skeleton of the stent.” The entire disclosure of U.S. Pat. No. 6,280,385, as it relates to the stent described by such claim 19, is hereby incorporated by reference into this specification.

Claim 20 of U.S. Pat. No. 6,280,385 describes “20. The stent according to claim 19, wherein the coil is connected to the skeleton in such a manner that it unfolds together with the skeleton when unfolding the stent.” The entire disclosure of U.S. Pat. No. 6,280,385, as it relates to the stent described by such claim 10, is hereby incorporated by reference into this specification.

Claim 21 of U.S. Pat. No. 6,280,385 describes “21. The stent according to claim 20, wherein the inductor comprises parallel conductors that partially act as a capacitor.” The entire disclosure of U.S. Pat. No. 6,280,385, as it relates to the stent described by such claim 21, is hereby incorporated by reference into this specification.

Claim 22 of U.S. Pat. No. 6,280,385 describes “22. The stent according to claim 20, wherein the capacitor comprises a separately provided condenser.” The entire disclosure of U.S. Pat. No. 6,280,385, as it relates to the stent described by such claim 22, is hereby incorporated by reference into this specification.

Claim 23 of U.S. Pat. No. 6,280,385 describes “23. The stent according to claim 22, wherein the stent comprises a detuning circuit for detuning the resonance circuit when applying the high-frequency radiation.” The entire disclosure of U.S. Pat. No. 6,280,385, as it relates to the stent described by such claim 23, is hereby incorporated by reference into this specification.

Claim 24 of U.S. Pat. No. 6,280,385 describes “24. The stent according to claim 23, wherein the detuning circuit comprises a condenser which is switchable parallel to the capacitor of the resonance circuit with the application of high-frequency radiation.” The entire disclosure of U.S. Pat. No. 6,280,385, as it relates to the stent described by such claim 24, is hereby incorporated by reference into this specification.

Claim 25 of U.S. Pat. No. 6,280,385 describes “25. The stent according to claim 24, wherein the switch circuit comprises two diodes which are switchable parallel to the capacitor.” The entire disclosure of U.S. Pat. No. 6,280,385, as it relates to the stent described by such claim 25, is hereby incorporated by reference into this specification.

Claim 26 of U.S. Pat. No. 6,280,385 describes “26. The stent according to claim 25, further comprises a switch coupled to activate or deactivate at least one resonance circuit.” The entire disclosure of U.S. Pat. No. 6,280,385, as it relates to the stent described by such claim 26, is hereby incorporated by reference into this specification.

Claim 27 of U.S. Pat. No. 6,280,385 describes “27. The stent according to claim 26, wherein at least one of the inductor and the capacitor of the resonance circuit are adjustable for the tuning of the resonance frequency of the magnetic resonance imaging system.” The entire disclosure of U.S. Pat. No. 6,280,385, as it relates to the stent described by such claim 27, is hereby incorporated by reference into this specification.

Claim 28 of U.S. Pat. No. 6,280,385 describes “28. The stent according to claim 27, wherein when a change in geometry of the stent occurs during its deployment, a product of the inductor and the capacitor of the resonance circuit remains approximately constant.” The entire disclosure of U.S. Pat. No. 6,280,385, as it relates to the stent described by such claim 28, is hereby incorporated by reference into this specification.

Claim 29 of U.S. Pat. No. 6,280,385 describes “29. The stent according to claim 28, wherein the resonance circuit has a low quality (Q factor), such that a broad frequency response is provided.” The entire disclosure of U.S. Pat. No. 6,280,385, as it relates to the stent described by such claim 29, is hereby incorporated by reference into this specification.

Claim 30 of U.S. Pat. No. 6,280,385 describes “30. The stent according to claim 29, wherein the resonance circuit has plural parallel switched inductors.” The entire disclosure of U.S. Pat. No. 6,280,385, as it relates to the stent described by such claim 30, is hereby incorporated by reference into this specification.

Claim 31 of U.S. Pat. No. 6,280,385 describes “31. The stent according to claim 29, wherein the resonance circuit has plural serially switched inductors.” The entire disclosure of U.S. Pat. No. 6,280,385, as it relates to the stent described by such claim 31, is hereby incorporated by reference into this specification.

Claim 32 of U.S. Pat. No. 6,280,385 describes “32. The stent according to claim 29, wherein the resonance circuit has plural parallel switched capacitors.” The entire disclosure of U.S. Pat. No. 6,280,385, as it relates to the stent described by such claim 32, is hereby incorporated by reference into this specification.

Claim 33 of U.S. Pat. No. 6,280,385 describes “33. The stent according to claim 29, wherein the resonance circuit has plural serially switched capacitors.” The entire disclosure of U.S. Pat. No. 6,280,385, as it relates to the stent described by such claim 33, is hereby incorporated by reference into this specification.

Claim 34 of U.S. Pat. No. 6,280,385 describes “34. The stent according to claim 23, wherein the detuning circuit comprises a coil which is switchable parallel to the inductance of the resonance circuit with the application of high-frequency radiation.” The entire disclosure of U.S. Pat. No. 6,280,385, as it relates to the stent described by such claim 34, is hereby incorporated by reference into this specification.

Claim 35 of U.S. Pat. No. 6,280,385 describes “35. The stent according to claim 22, further comprising a switch circuit coupled to short circuit the capacitor when applying the high-frequency radiation.” The entire disclosure of U.S. Pat. No. 6,280,385, as it relates to the stent described by such claim 35, is hereby incorporated by reference into this specification.

A Biocompatible Stent Assembly

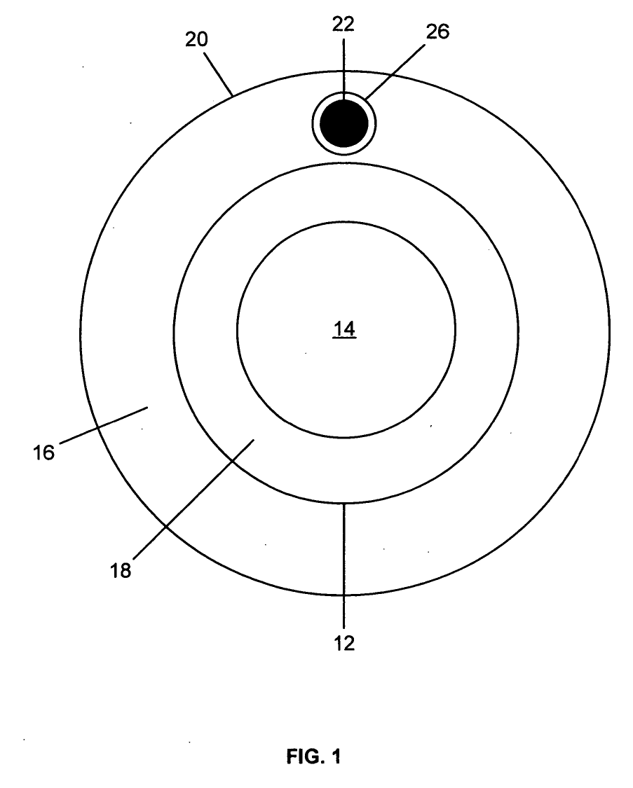

FIG. 1 is a schematic sectional view of a stent assembly 10 that, in one preferred embodiment thereof, is biocompatible.

Referring to FIG. 1, and to the preferred embodiment depicted therein, it will be seen that stent assembly 10 is preferably comprised of a stent 12 comprised of a lumen 14. As used in this specification, lumen means the interior of the stent, and more particularly, the interior of the volume defined by the stent's structure. The stent 12 may be any of the stents described in the prior art.

In one preferred embodiment, the stent 12 is similar in structure to one or more of the stents disclosed in published United States patent application 2004/0030379, the entire disclosure of which is hereby incorporated by reference into this specification. Thus, and referring to page 4 of such published patent application, “Medical devices which are particularly suitable for the present invention include any kind of stent for medical purposes, which are known to the skilled artisan. Suitable stents include, for example, vascular stents such as self-expanding stents and balloon expandable stents. Examples of self-expanding stents useful in the present invention are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 4,733,665 issued to Palmaz, U.S. Pat. No. 4,800,882 issued to Gianturco, U.S. Pat. No. 4,886,062 issued to Wiktor and U.S. Pat. No. 5,449,373 issued to Pinchasik et al. A bifurcated stent is also included among the medical devices suitable for the present invention.”

The stent 12 may be made from metallic materials, and/or polymeric materials. As is also disclosed in published United States patent application 2004/0030379. “The medical devices suitable for the present invention may be fabricated from polymeric and/or metallic materials. Examples of such polymeric materials include polyurethane and its copolymers, silicone and its copolymers, ethylene vinyl-acetate, poly(ethylene terephthalate), thermoplastic elastomer, polyvinyl chloride, polyolephines, cellulosics, polyamides, polyesters, polysulfones, polytetrafluoroethylenes, acrylonitrile butadiene styrene copolymers, acrylics, polyactic acid, polyclycolic acid, polycaprolactone, polyacetal, poly(lactic acid), polylactic acid-polyethylene oxide copolymers, polycarbonate cellulose, collagen and chitins. Examples of suitable metallic materials include metals and alloys based on titanium (e.g., nitinol, nickel titanium alloys, thermo-memory alloy materials), stainless steel, platinum, tantalum, nickel-chrome, certain cobalt alloys including cobalt-chromium-nickel alloys (e.g., Elgiloy® and Phynox®) and gold/platinum alloy. Metallic materials also include clad composite filaments, such as those disclosed in WO 94/16646.”

By way of further illustration, the stent 12 may be a drug-eluting intravascular stent. Thus, e.g., and as is disclosed in U.S. Pat. Nos. 5,591,227, 5,599,352, and 6,597,967 (the entire disclosure of each of which is hereby incorporated by reference into this specification), the medical device may be “. . . a drug eluting intravascular stent comprising: (a) a generally cylindrical stent body; (b) a solid composite of a polymer and a therapeutic substance in an adherent layer on the stent body; and (c) fibrin in an adherent layer on the composite.”

By way of yet further illustration, and as is disclosed in U.S. Pat. No. 6,623,521 (the entire disclosure of which is hereby incorporated by reference into this specification), the stent 12 may be an expandable stent with sliding and locking radial elements. This patent discloses many other “prior art” stents, whose designs also may be utilized as stent 12. Thus as is disclosed at columns 1-2 of this patent, “Examples of prior developed stents have been described by Balcon et al., “Recommendations on Stent Manufacture, Implantation and Utilization,” European Heart Journal (1997), vol. 18, pages 1536-1547, and Phillips, et al., “The Stenter's Notebook,” Physician's Press (1998), Birmingham, Mich. The first stent used clinically was the self-expanding “Wallstent” which comprised a metallic mesh in the form of a Chinese fingercuff. This design concept serves as the basis for many stents used today. These stents were cut from elongated tubes of wire braid and, accordingly, had the disadvantage that metal prongs from the cutting process remained at the longitudinal ends thereof. A second disadvantage is the inherent rigidity of the cobalt based alloy with a platinum core used to form the stent, which together with the terminal prongs, makes navigation of the blood vessels to the locus of the lesion difficult as well as risky from the standpoint of injury to healthy tissue along the passage to the target vessel. Another disadvantage is that the continuous stresses from blood flow and cardiac muscle activity create significant risks of thrombosis and damage to the vessel walls adjacent to the lesion, leading to restenosis. A major disadvantage of these types of stents is that their radial expansion is associated with significant shortening in their length, resulting in unpredictable longitudinal coverage when fully deployed.”

Other “prior art stents” which may be used as stent 12 are also disclosed in U.S. Pat. No. 6,623,521. As is also disclosed in U.S. Pat. No. 6,623,521 “Among subsequent designs, some of the most popular have been the Palmaz-Schatz slotted tube stents. Originally, the Palmaz-Schatz stents consisted of slotted stainless steel tubes comprising separate segments connected with articulations. Later designs incorporated spiral articulation for improved flexibility. These stents are delivered to the affected area by means of a balloon catheter, and are then expanded to the proper size. The disadvantage of the Palmaz-Schatz designs and similar variations is that they exhibit moderate longitudinal shortening upon expansion, with some decrease in diameter, or recoil, after deployment. Furthermore, the expanded metal mesh is associated with relatively jagged terminal prongs, which increase the risk of thrombosis and/or restenosis. This design is considered current state of the art, even though their thickness is 0.004 to 0.006 inches.”

Other “prior art stents” which may be used as stent 12 are also disclosed in U.S. Pat. No. 6,623,521. As is also disclosed in U.S. Pat. No. 6,623,521, “Another type of stent involves a tube formed of a single strand of tantalum wire, wound in a sinusoidal helix; these are known as coil stents. They exhibit increased flexibility compared to the Palnaz-Schatz stents. However, they have the disadvantage of not providing sufficient scaffolding support for many applications, including calcified or bulky vascular lesions. Further, the coil stents also exhibit recoil after radial expansion.”

Other “prior art stents” which may be used as stent 12 are also disclosed in U.S. Pat. No. 6,623,521. As is also disclosed in U.S. Pat. No. 6,623,521, “One stent design described by Fordenbacher, employs a plurality of elongated parallel stent components, each having a longitudinal backbone with a plurality of opposing circumferential elements or fingers. The circumferential elements from one stent component weave into paired slots in the longitudinal backbone of an adjacent stent component. By incorporating locking means within the slotted articulation, the Fordenbacher stent may minimize recoil after radial expansion. In addition, sufficient numbers of circumferential elements in the Fordenbacher stent may provide adequate scaffolding. Unfortunately, the free ends of the circumferential elements, protruding through the paired slots, may pose significant risks of thrombosis and/or restenosis. Moreover, this stent design would tend to be rather inflexible as a result of the plurality of longitudinal backbones.”

Other “prior art stents” which may be used as stent 12 are also disclosed in U.S. Pat. No. 6,623,521. As is also disclosed in U.S. Pat. No. 6,623,521, “Some stents employ “jelly roll” designs, wherein a sheet is rolled upon itself with a high degree of overlap in the collapsed state and a decreasing overlap as the stent unrolls to an expanded state. Examples of such designs are described in U.S. Pat. No. 5,421,955 to Lau, U.S. Pat. Nos. 5,441,515 and 5,618,299 to Khosravi, and U.S. Pat. No. 5,443,500 to Sigwart. The disadvantage of these designs is that they tend to exhibit very poor longitudinal flexibility. In a modified design that exhibits improved longitudinal flexibility, multiple short rolls are coupled longitudinally. See e.g., U.S. Pat. No. 5,649,977 to Campbell and U.S. Pat. Nos. 5,643,314 and 5,735,872 to Carpenter. However, these coupled rolls lack vessel support between adjacent rolls.”

Other “prior art stents” which may be used as stent 12 are also disclosed in U.S. Pat. No. 6,623,521. As is also disclosed in U.S. Pat. No. 6,623,521, “Another form of metal stent is a heat expandable device using Nitinol or a tin-coated, heat expandable coil. This type of stent is delivered to the affected area on a catheter capable of receiving heated fluids. Once properly situated, heated saline is passed through the portion of the catheter on which the stent is located, causing the stent to expand. The disadvantages associated with this stent design are numerous. Difficulties that have been encountered with this device include difficulty in obtaining reliable expansion, and difficulties in maintaining the stent in its expanded state.”

Other “prior art stents” which may be used as stent 12 are also disclosed in U.S. Pat. No. 6,623,521. As is also disclosed in U.S. Pat. No. 6,623,521, “Self-expanding stents are also available. These are delivered while restrained within a sleeve (or other restraining mechanism), that when removed allows the stent to expand. Self-expanding stents are problematic in that exact sizing, within 0.1 to 0.2 mm expanded diameter, is necessary to adequately reduce restenosis. However, self-expanding stents are currently available only in 0.5 mm increments. Thus, greater selection and adaptability in expanded size is needed.”

The stent 12 may also be the stent design claimed in U.S. Pat. No. 6,623,521. This stent design “An expandable intraluminal stent, comprising: a tubular member comprising a clear through-lumen, and having proximal and distal ends and a longitudinal length defined there between, a circumference, and a diameter which is adjustable between at least a first collapsed diameter and at least a second expanded diameter, said tubular member comprising: at least one module comprising a series of radial elements, wherein each radial element defines a portion of the circumference of the tubular member and wherein no radial element overlaps with itself in either the first collapsed diameter or the second expanded diameter; at least one articulating mechanism which permits one-way sliding of the radial elements from the first collapsed diameter to the second expanded diameter, but inhibits radial recoil from the second expanded diameter; and a frame element which surrounds at least one radial element in each module.”

By way of yet further illustration, the stent 12 may be the multi-coated drug-eluting stent described in U.S. Pat. No. 6,702,850, the entire disclosure of which is hereby incorporated by reference in to this specification. This patent describes and claims: “. . . a stent body comprising a surface; and a coating comprising at least two layers disposed over at least a portion of the stent body, wherein the at least two layers comprise a first layer disposed over the surface of the stent body and a second layer disposed over the first layer, said first layer comprising a polymer film having a biologically active agent dispersed therein, and the second layer comprising an antithrombogenic heparinized polymer comprising a macromolecule, a hydrophobic material, and heparin bound together by covalent bonds, wherein the hydrophobic material has more than one reactive functional group and under 100 mg/ml water solubility after being combined with the macromolecule.”

By way of yet further illustration, the stent 12 may be one or more of the coronary stents disclosed in Patrick W. Serruys “Handbook of Coronary Stents,” Fourth Edition (Martin Dunitz Ltd, London, England, 2002). Thus, and referring to such book, the stent 12 may be the “ARTHOS” stent (which contains a stent surface which blocks ion diffusion from its stainless steel material), the “ANTARES STARFLEX” stent (a homogeneous, multicellular stent structure with alternating stiff and flex segments), the “SLK-VIEW” stent (a 316 L stainless steel flexible slotted tube stent with a side aperture located between the proximal and distal section), the “BeStent2” stent (a stainless steel stent with solid gold radiopaque end markers), the “BiodivYsio” stent (a stent coated with phosphorylcholine), the “Carbostent SIRIUS” stent (a stent coated with pure turbostratic carbon), the “Corodynamic APOLO” stent (a segmented multicellular slotted tube with alternating bridge connections), the “COROFLEX” coronary stent (a laser-cut, 316 L stainless steel slotted-tube which has rounded edges and is electropolished), the “DURAFLEX” coronary stent (a laser-cut, stainless steel stent having circumferential rings linked by flexible cross bridges), the “EXPRESS” coronary stent system (an expandable stent comprised of multiple rings connected with multiple links), the “GENIC DYLYN” stent (an expandable coronary stent with a helical sinusoidal waveform geometry), the “IGAKI-TAMAI” stent (a biodegradable stent made of poly-L-lactic acid that has a zigzag helical coil design), the “JOSTENT” coronary stent (a coil stent with spiral links), the “JOSTENT B]OFLEX” stent (a super-elastic Nitinol stent based upon a slotted tube design), the “LUNAR” coronary stent (a homogeneous, multicellular stent structure with alternating stiff and flex segments made of Niobium alloy coated with iridium oxide), the “MANEO” stent (a multicellular stent whose segments are connected with multiple links), the “MEDTRONIC AVE MODULAR” stent (a balloon-expandable stent with ellipto-rectangular struts), the “PENTA” coronary stent (a stent comprised of multiple rings connected with multiple links), the “NEXUS” coronary stent (a balloon expandable stent with multiple cells and multiple “V” connectors), the “PROLINK” stent (a stent with a corrugated, ultrathin ring design wherein the thin rings are interconnected by three alternating links), the “PROPASS” stent (a platinum activated stent with a platinum coating), the “RITHRON” coronary stent (a flexible and conformable stent coated with a thin, hypothrombogenic ocating of amorphous huydrogenated silicon carbide), the “SPIRAL FORCE” stent (a tubular stent in which all of the struts are connected with inverted C-joints), the “TSUNAMI” coronary stent (a stent with a double-link structure in which diamond-shaped cells are joined by two connectors), and the like.

By way of yet further illustration, the stent 12 may be one or more of the drug-eluting stents described at pages 285-366 of Patrick W. Serruys “Handbook of Coronary Stents, supra. Thus, e.g., the stent 12 may be a “BIODIVYSIO MATRIX” stent (a stent coated with a coating with a molecular weight less than 1200 daltons, a Boston Scientific “TAXUS” stent (a stent with a proprietary copolymer carrier system comprised of Paclitaxel), the multi-link “TETRA-D” stent (a stent adapted to elute Actinomycin D, an antibiotic that has been approved for clinical use as an anti-cancer agent), the “PHYTIS” double-coated stent (a stent that elutes 17-beta-estradiol and is comprised of a diamond-like carbon coating), the “QUADDS” stent (a stent covered with a series of 2 mm. polymer sleeves made from an acrylate polymer and formed into ringed sleeves), the “BX VELOCITY” stent (a stent coated with a thin layer of non-erodable methacrylate and an ethylene-based copolymer), and the like.

By way of further illustration, the stent 12 may be one or more of the drug-eluting stents described and/or claimed in U.S. Pat. Nos. 5,591,227, 5,999,352, and 5,697,967, the entire disclosure of each of which is hereby incorporated by reference into this specification. U.S. Pat. No. 5,591,227 claims, in claim 1, “1. A drug eluting intravascular stent comprising: (a) a generally cylindrical stent body; (b) a solid composite of a polymer and a therapeutic substance in an adherent layer on the stent body; and(c) fibrin in an adherent layer on the composite.”

U.S. Pat. No. 6,702,850, referred to elsewhere in this specification, contains an excellent discussion of drug-eluting stent technology. The entire disclosure of U.S. Pat. No. 6,702,850 is hereby incorporated by reference into this specification.

In the introductory portion of U.S. Pat. No. 6,702,850, it is disclosed that “This invention relates to coated stents for carrying biologically active agents to provide localized treatment at the implant site and methods of applying stent coatings. In particular, this invention relates to antithrombogenic and antirestenotic stents having a multi-layered coating, wherein the first or inner layer is formed from a polymer and one or more biologically active agents, and a second or outer layer is formed from an antithrombogenic heparinized polymer. This invention also relates to methods of applying a multi-layer coating over the surface of a stent and methods of using such a coated stent.”

U.S. Pat. No. 6,702,850 also discloses that “An important consideration in using coated stents is the release rate of the drug from the coating. It is desirable that an effective therapeutic amount of the drug be released from the stent for the longest period of time possible. Burst release, a high release rate immediately following implantation, is undesirable and a persistent problem. While typically not harmful to the patient, a burst release “wastes” the limited supply of the drug by releasing several times the effective amount required and shortens the duration of the release period. Several techniques have been developed in an attempt to reduce burst release. For example, U.S. Pat. No. 6,258,121 B1 to Yang et al. discloses a method of altering the release rate by blending two polymers with differing release rates and incorporating them into a single layer.”

U.S. Pat. No. 6,702,850 also discloses that “Heparin, generally derived from swine intestine, is a substance that is well known for its anticoagulation ability. It is known in the art to apply a thin polymer coating loaded with heparin onto the surface of a stent using the solvent evaporation technique. For example, U.S. Pat. No. 5,837,313 to Ding et al. describes a method of preparing a heparin coating composition.”

U.S. Pat. No. 6,702,850 also discloses that “In view of the foregoing, it will be appreciated that the development of a stent having a multi-layered coating, where one layer comprises a thin film of polymeric material with a biologically active agent dispersed therein, and a second layer is disposed over the first layer where the second layer comprises a hydrophobic heparinized polymer, would be a significant advance in the art. It will also be appreciated that the current invention inhibits both restenosis and thrombosis, and can be effective in delivering a wide range of other therapeutic agents to the implant site over a relatively extended period of time.”

Any of the biologically active agents described in U.S. Pat. No. 6,702,850 may be used in the stent of the instant invention. As is disclosed in U.S. Pat. No. 6,702,850, “As used herein, ‘biologically active agent’ means a drug or other substance that has therapeutic value to a living organism including without limitation antithrombotics, anticoagulants, antiplatelet agents, thrombolytics, antiproliferatives, anti-inflammatories, agents that inhibit restenosis, smooth muscle cell inhibitors, antibiotics, and the like, and mixtures thereof.”

Thus, e.g., one may use any of the anticancer drugs disclosed in U.S. Pat. No. 6,702,850 in the stent 12 of this invention. As is disclosed in such U.S. patent, “Illustrative anticancer drugs include acivicin, aclarubicin, acodazole, acronycine, adozelesin, alanosine, aldesleukin, allopurinol sodium, altretamine, aminoglutethimide, amonafide, ampligen, amsacrine, androgens, anguidine, aphidicolin glycinate, asaley, asparaginase, 5-azacitidine, azathioprine, Bacillus calmette-guerin (BCG), Baker's Antifol (soluble), beta-2′-deoxythioguanosine, bisantrene hcl, bleomycin sulfate, busulfan, buthionine sulfoximine, BWA 773U82, BW 502U83.HCl, BW 7U85 mesylate, ceracemide, carbetimer, carboplatin, carmustine, chlorambucil, chloroquinoxaline-sulfonamide, chlorozotocin, chromomycin A3, cisplatin, cladribine, corticosteroids, Corynebacterium parvum, CPT-11, crisnatol, cyclocytidine, cyclophosphamide, cytarabine, cytembena, dabis maleate, dacarbazine, dactinomycin, daunorubicin HCl, deazauridine, dexrazoxane, dianhydrogalactitol, diaziquone, dibromodulcitol, didemnin B, diethyldithiocarbamate, diglycoaldehyde, dihydro-5-azacytidine, doxorubicin, echinomycin, edatrexate, edelfosine, eflomithine, Elliott's solution, elsamitrucin, epirubicin, esorubicin, estramustine phosphate, estrogens, etanidazole, ethiofos, etoposide, fadrazole, fazarabine, fenretinide, filgrastim, finasteride, flavone acetic acid, floxuridine, fludarabine phosphate, 5-fluorouracil, Fluosol®, flutamide, gallium nitrate, gemcitabine, goserelin acetate, hepsulfam, hexamethylene bisacetamide, homoharringtonine, hydrazine sulfate, 4-hydroxyandrostenedione, hydrozyurea, idarubicin HCl, ifosfamide, interferon alfa, interferon beta, interferon gamma, interleukin-1 alpha and beta, interleukin-3, interleukin-4, interleukin-6, 4-ipomeanol, iproplatin, isotretinoin, leucovorin calcium, leuprolide acetate, levamisole, liposomal daunorubicin, liposome encapsulated doxorubicin, lomustine, lonidamine, maytansine, mechlorethamine hydrochloride, melphalan, menogaril, merbarone, 6-mercaptopurine, mesna, methanol extraction residue of Bacillus calmette-guerin, methotrexate, N-methylformamide, mifepristone, mitoguazone, mitomycin-C, mitotane, mitoxantrone hydrochloride, monocyte/macrophage colony-stimulating factor, nabilone, nafoxidine, neocarzinostatin, octreotide acetate, ormaplatin, oxaliplatin, paclitaxel, pala, pentostatin, piperazinedione, pipobroman, pirarubicin, piritrexim, piroxantrone hydrochloride, PIXY-321, plicamycin, porfimer sodium, prednimustine, procarbazine, progestins, pyrazofurin, razoxane, sargramostim, semustine, spirogermanium, spiromustine, streptonigrin, streptozocin, sulofenur, suramin sodium, tamoxifen, taxotere, tegafur, teniposide, terephthalamidine, teroxirone, thioguanine, thiotepa, thymidine injection, tiazofurin, topotecan, toremifene, tretinoin, trifluoperazine hydrochloride, trifluridine, trimetrexate, tumor necrosis factor, uracil mustard, vinblastine sulfate, vincristine sulfate, vindesine, vinorelbine, vinzolidine, Yoshi 864, zorubicin, and mixtures thereof.”

Thus, e.g., one may use any of the antiflammatory drugs disclosed in U.S. Pat. No. 6,702,850 in the stent 12 of this invention. As is disclosed in such United States patent, “Illustrative antiinflammatory drugs include classic non-steroidal anti-inflammatory drugs (NSAIDS), such as aspirin, diclofenac, indomethacin, sulindac, ketoprofen, flurbiprofen, ibuprofen, naproxen, piroxicam, tenoxicam, tolmetin, ketorolac, oxaprosin, mefenamic acid, fenoprofen, nambumetone (relafen), acetaminophen (Tylenol®), and mixtures thereof; COX-2 inhibitors, such as nimesulide, NS-398, flosulid, L-745337, celecoxib, rofecoxib, SC-57666, DuP-697, parecoxib sodium, JTE-522, valdecoxib, SC-58125, etoricoxib, RS-57067, L-748780, L-761066, APHS, etodolac, meloxicam, S-2474, and mixtures thereof; glucocorticoids, such as hydrocortisone, cortisone, prednisone, prednisolone, methylprednisolone, meprednisone, triamcinolone, paramethasone, fluprednisolone, betamethasone, dexamethasone, fludrocortisone, desoxycorticosterone, and mixtures thereof; and mixtures thereof.”

One may use one or more of the drug-eluting polymers disclosed in U.S. Pat. No. 6,702,850 in stent 12. Thus, and as is disclosed in such patent, “In an illustrative embodiment, the first layer comprises a polymeric film loaded with a biologically active agent that prevents smooth cell proliferation, such as echinomycin. Illustrative polymers that can be used for making the polymeric film include polyurethanes, polyethylene terephthalate (PET), PLLA-poly-glycolic acid (PGA) copolymer (PLGA), polycaprolactone (PCL) poly-(hydroxybutyrate/hydroxyvalerate) copolymer (PHBV), poly(vinylpyrrolidone) (PVP), polytetrafluoroethylene (PTFE, Teflon™), poly(2-hydroxyethylmethacrylate) (poly-HEMA), poly(etherurethane urea), silicones, acrylics, epoxides, polyesters, urethanes, parlenes, polyphosphazene polymers, fluoropolymers, polyamides, polyolefins, and mixtures thereof. The second layer comprises a hydrophobic heparinized polymer with strong anticoagulation properties. The second layer of the hydrophobic heparinized polymer also has the effect of preventing a burst release of the biologically active agent dispersed in the first layer—resulting in a relatively longer release period of the biologically active agent. It should also be understood that the first layer can contain more than one biologically active agent.

The stent 12 may be any of the metal stents disclosed in U.S. Pat. No. 6,702,850. Thus, as is disclosed in such patent, “The style and composition of the stent may comprise any biocompatible material having the ability to support a diseased vessel. In general, it is preferred to use a metal stent, such as those manufactured from stainless steel, gold, titanium or the like, but plastic or other appropriate materials may be used. In one preferred embodiment, the stent is a Palmz-Schatz stent manufactured by Cordis Corp. (Miami, Fla.). The stent may be self expanding or balloon expanding. It is preferred that the coating substantially cover the entire stent surface, but it is within the scope of this invention to have the coating cover only a portion of the stent. It is also to be understood that any substrate, medical device, or part thereof having contact with organic fluid, or the like, may also be coated.”

The stent 12 may comprise one or more of the antithromogenic agents disclosed in U.S. Pat. No. 6,702,850. Thus, as is disclosed in such patent, “The second layer of the stent coating comprises an antithrombogenic heparinized polymer. Antithrombogenic heparinized polymers are soluble only in organic solvents and are insoluble in water. Antithrombogenic heparin polymers are produced by binding heparin to macromolecules and hydrophobic materials.”

The stent 12 may comprise one or more of the macromolecules disclosed in U.S. Pat. No. 6,702,850. Thus, and as is disclosed in such patent, “Illustrative macromolecules include synthetic macromolecules, proteins, biopolymers, and mixtures thereof. Illustrative synthetic macromolecules include polydienes, polyalkenes, polyacetylenes, polyacrylic acid and its derivatives, poly α-substituted acrylic acid and its derivatives, polyvinyl ethers, polyvinylalcohol, polyvinyl halides, polystyrene and its derivatives, polyoxides, polyethers, polyesters, polycarbonates, polyamides, polyamino acids, polyureas, polyurethanes, polyimines, polysulfides, polyphosphates, polysiloxanes, polysilsesquioxanes, polyheterocyclics, cellulose and its derivatives, and polysaccharides and their copolymers or derivatives. Illustrative proteins that can be used according to the present invention include protamine, polylysine, polyaspartic acid, polyglutamic acid, and derivatives and copolymers thereof. Illustrative biopolymers that can be used according to the present invention include polysaccharides, gelatin, collagen, alginate, hyaluronic acid, alginic acid, carrageenan, chondroitin, pectin, chitosan, and derivatives and copolymers thereof.”

Referring again to FIG. 1, the stent 12 may comprise one or more of the drug-eluting polymers known to those skilled in the art. These drug eluting polymers may be present as drug eluting polymer layer 16, which is preferably disposed on the top surface of stent 12. Alternatively, and/or preferably additionally, the drug eluting polymer(s) may be present as drug eluting polymer 18, which is preferably disposed between lumen 14 and the bottom layer of the stent.

Thus, for example, one may use one or more of the drug-eluting polymers disclosed in U.S. Pat. No. 5,545,208, the entire disclosure of which is hereby incorporated by reference into this specification. As is disclosed in such patent, “Several polymeric compounds that are known to be bioabsorbable and hypothetically have the ability to be drug impregnated may be useful in prosthesis formation herein. These compounds include: poly-1-lactic acid/polyglycolic acid, polyanhydride, and polyphosphate ester. A brief description of each is given below.”

As is also disclosed in U.S. Pat. No. 5,545,208, “Poly-1-lactic acid/polyglycolic acid has been used for many years in the area of bioabsorbable sutures. It is currently available in many forms, i.e., crystals, fibers, blocks, plates, etc. These compounds degrade into non-toxic lactic and glycolic acids. There are, however, several problems with this compound. The degradation artifacts (lactic acid and glycolic acid) are slightly acidic. The acidity causes minor inflammation in the tissues as the polymer degrades. This same inflammation could be very detrimental in coronary and peripheral arteries, i.e., vessel occlusion. Another problem associated with this polymer is the ability to control and predict the degradation behavior. It is not possible for the biochemist to safely predict degradation time. This could be very detrimental for a drug delivery device.”

As is also disclosed in U.S. Pat. No. 5,545,208, “Another compound which could be used are the polyanhydrides. They are currently being used with several chemotherapy drugs for the treatment of cancerous tumors. These drugs are compounded into the polymer which is molded into a cube-like structure and surgically implanted at the tumor site.”

As is also disclosed in U.S. Pat. No. 5,545,208, “The compound which is preferred is a polyphosphate ester. Polyphosphate ester is a compound such as that disclosed in U.S. Pat. Nos. 5,176,907; 5,194,581; and 5,656,765 issued to Leong which are incorporated herein by reference. Similar to the polyanhydrides, polyphoshate ester is being researched for the sole purpose of drug delivery. Unlike the polyanhydrides, the polyphosphate esters have high molecular weights (600,000 average), yielding attractive mechanical properties. This high molecular weight leads to transparency, and film and fiber properties. It has also been observed that the phosphorous-carbon-oxygen plasticizing effect, which lowers the glass transition temperature, makes the polymer desirable for fabrication.”

By way of further illustration, one may use one or more of the drug eluting materials disclosed in U.S. Pat. No. 4,953,564 (screw-in drug eluting stent), U.S. Pat. No. 5,217,028 (bipolar cardiac lead with drug eluting device), U.S. Pat. No. 5,545,208 (intralumenal drug eluting prosthesis), U.S. Pat. No. 5,591,227 (drug eluting stent), U.S. Pat. No. 5,599,352 (metnhod of making a drug eluting stent), U.S. Pat. No. 5,697,967 (drug eluting stent), U.S. Pat. No. 5,725,567 (method of making an intralumenal drug eluting prosthesis), U.S. Pat. No. 5,851,217 (intralumenal drug eluting prosthesis), U.S. Pat. No. 5,851,231 (intralumenal drug eluting prosthesis), U.S. Pat. No. 5,871,535 (intralumenal drug eluting prosthesis), U.S. Pat. No. 6,004,346 (intralumenal drug eluting prosthesis), U.S. Pat. No. 6,206,914 (implantable system with drug eluting cells for on-demand drug delivery), U.S. Pat. No. 6,671,562 (high impedance drug eluting cardiac lead), U.S. Pat. No. 6,716,444 (barriers for polymer-coated implantable medical devices), U.S. Pat. No. 6,824,561 (implantable system with drug eluting cells for on-demand drug delivery), U.S. Pat. No. 6,830,747 (biodegradable copolymers linked to segment with a plurality of functional groups), and the like. The entire disclosure of each of these United States patents is hereby incorporated by reference into this specification.

By way of further illustration, one may use one or more of the drug-eluting polymers disclosed in Table 8 (at page 166) of J. R. Davis' “Handbook of Materials for Medical Devices” (ASM International, Materials Park, Ohio, 2003). These polymers include natural polymers such as, e.g., cellulose acetate phthalate, hydroxypropyl cellulose, carboxymethylcellulose, ethyl cellulose, methyl cellulose, collagen, zein, gelatin, natural rubber, guar gum, gum agar, and albumin. These polymers also include synthetic elastomeric polymers such as, e.g., silicone rubber, polysiloxane, polybutadiene, and polyisoprene. These polymers also include synthetic hydrogels such as,e.g., polyhydroxyalkyl methacrylates, polyvinyl alcohol, polyvinyl pyrrolidone, aligantes, and polyacrylamide. These polymers also include synthetic biodegradable polymers such as, e.g., polylactic acid, polyglycolic acid, polyalkyl 2-cyanoacrylates, polyurethanes, polyanhydrides, pand polyorthoesters. These polymers also include synthetic adhesives such as, e.g., polyisobutylenes, polacrylates, and silicones. These polymers also include others materials such as, e.g., polyvinyl chloride, polyvinyl acetate, ethylene-vinyl acetate, polyethylene, and polyurethanes. Reference also may be had, e.g., to a work by R. Toddywala et al., “Polymers for Controlled Drug Delivery, Concise Encylopedia of Medical and Dental Materials, D. F. Williams, editor (Pergamon Press and the MIT Press, 1990), at pages 280-289.

Referring again to FIG. 1, and inductor assembly 20 comprised of an inductor 22 is either disposed on or over the stent 12. This inductor 22 may be similar to, or indentical to, the inductor disclosed in U.S. Pat. No. 6,280,385, the entire disclosure of which is hereby incorporated by reference into this specification. As used in this specification, inductor means a circuit component designed so that inductance is its most important property. Electronic Dictionary (1st edition), Cooke and Marcus, McGraw-Hill Book Company, Inc. (1945) p. 179.

Alternatively, the inductor 22 may be formed as an integral portion of the stent, as is also disclosed in U.S. Pat. No. 6,280,385. Selected portions of such patent will be quoted below to illustrate typical inductors 22 that may be used in the device 10 of this invention.

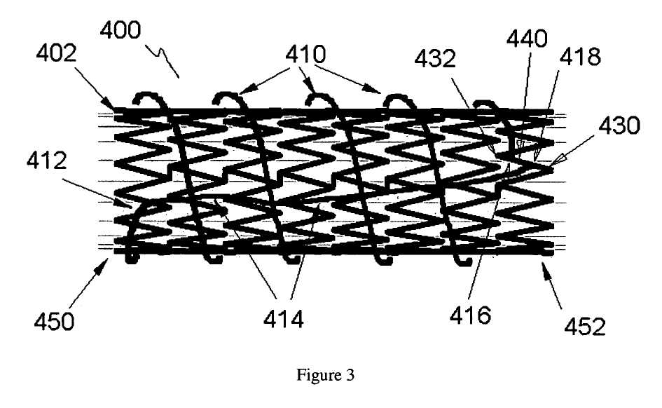

Column 7 of U.S. Pat. No. 6,280, 385 discloses an embodiment wherein the inductor 22 is an integral part of the stent assembly 12. “For improved imaging and functional control of the stent in the magnetic resonance image, the stent 1 according to the present invention and as shown in FIG. 1 is provided with an inductor defined by the skeleton 2 and a capacitor 3. Thus, the inductance of the stent 1 is provided by the skeleton 2 of the of the stent 1. Provision is made that the individual components of the skeleton 2 are insulated relative to each other as shown in FIG. 3. Insulation of the individual components of the skeleton 2 may take place during the manufacturing process, whereby an insulating layer is applied to the skeleton which is formed during separate phases of the manufacturing process of the stent which is made from a metal pipe or tube.” Referring to FIG. 1, and to the preferred embodiment depicted therein, applicants utilize a biocompatible material as the “insulating material 24.” This biocompatible material 24 will be discussed elsewhere in this specification.

U.S. Pat. No. 6,280,385 also disclose that “The inductor 2 is electrically connected to the capacitor 3, such that the inductor 2 and capacitor 3 form a resonance circuit. In FIG. 3 the capacitor 3 is provided as a plate capacitor defined by two plates 31 and 32. However, any other desired capacitor may be used. It is within the framework of this invention that the capacitor 3 does not represent an individual component, but that is consists simply of the inductor 2 from the material of the stent 1, e.g., it is formed by parallel wires of the wire skeleton. We may add, that for reasons of clearer depiction, the electrical connection between the capacitor plate 32 and the inductance is not shown in FIG. 1. “An inductor 22 that: . . . is formed by parallel wires of the wire skeleton . . . ” is within the scope of the instant invention.

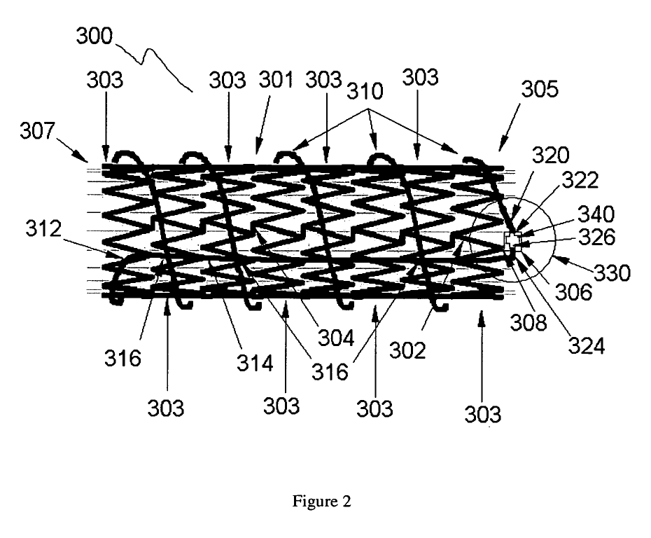

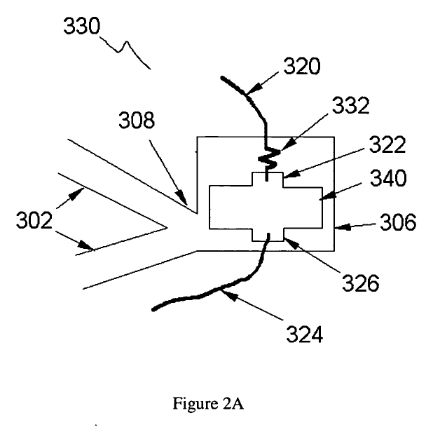

The inductor 22 may comprise one or more parallel switched inductors and/or serially switched inductances. Thus, and as is disclosed in Column 8 of U.S. Pat. No. 6,280,385, “The resonance circuit 4 can be designed in a multitude of embodiments. According to FIG. 2c, it may have several parallel switched inductances 2a to 2n and according to FIG. 2d it may have several parallel switched capacitors 3a to 3n. Furthermore, several inductances and/or capacitances may be serially switched. Several resonance circuits may also be provided on a stent which may each have a switch and may have serially and/or parallel switched inductors and/or capacitors. Especially with several parallel or serially switched inductances, flow measurements may be refined by means of suitable sequences.”

The inductor 22 (see FIG. 1) may be variable such that, as the configuration of the stent changes, the product of the inductance 22 and the capacitance of the assembly 10 is constant. One may use, e.g., the device described in lines 51 et seq. of Column 8 of U.S. Pat. No. 6,280,385, wherein it is disclosed that “A second variant provides an apparatus with the capability to keep the product of inductance and capacitance constant even after a change of the geometry as was observed in the example referring to the unfolding of the stent. This may take place either in that the stent is given a geometry that changes its properties as little as possible after unfolding of the stent. Thus, the stent is provided with a substantially constant inductance and a substantially constant capacitance. A widening of the stent at the implantation location thus essentially effects substantially no change in the resonance of the resonance circuit.”

As is also disclosed in the paragraph beginning at lines 63 of Column 8, “A constancy of the product of inductance and capacitance may be realized, among other things, by a compensation of the changing inductance by a correspondingly changing capacitance. For instance, provision is made that a capacitor surface is enlarged or decreased for compensation of a changing inductance by a correspondingly changing capacitance, such that the capacitance increases or decreases according to the corresponding distance of the capacitor surfaces. The movability of the capacitor plate 32 with regard to the capacitor plate 31 and the adjustability of the capacitance thereby is schematically shown in FIG. 1 by a double arrow.”

As is also disclosed in U.S. Pat. No. 6,280,385 (see the paragraph beginning at line 7 of Column 9), “A third variant discloses that an adjustment of the resonance circuit in the magnetic field of the nuclear spin tomograph is induced by a change or adjustment of the inductor and/or the capacitor of the resonance circuit after their placement. For example, a change of the capacitor surface is provided by means of the application instrument located in the body, such as a catheter. A decrease in the inductance and thus an adjustment of the resonance circuit to the resonance frequency in the nuclear spin tomograph may take place, for instance, by a laser induced mechanical or electrolytic insulation of coil segments. A change in the capacitor may also take place by a laser induced mechanical or electrolytic insulation of the capacitor.”

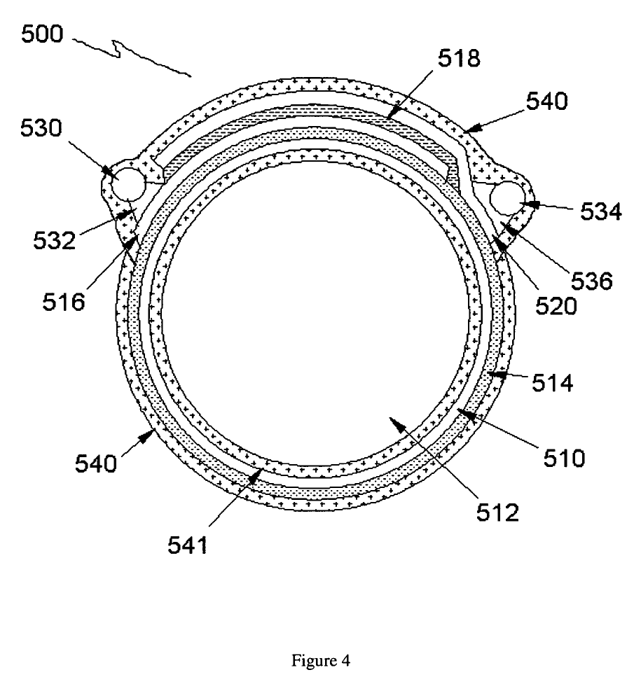

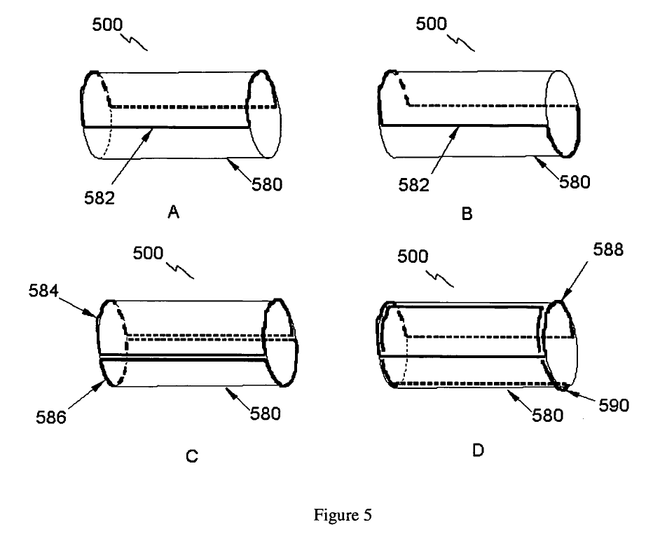

FIG. 3 of U.S. Pat. No. 6,280,385 discloses the preparation of a stent with a “layer 82“from which an inductor may be formed. As is disclosed in Column 9 of U.S. Pat. No. 6,280,385, “FIG. 3 schematically discloses a possible embodiment of a stent according to FIG. 1. According to FIGS. 4a and 4b, the stent material consists of two (FIG. 4a) or more (FIG. 4b) layers 81 and 82. The first layer 81 depicts the material for the actual stent function. It has poor conductivity and a high level of stability and elasticity. Suitable materials are mainly nickel-titanium, plastic or carbon fibers. The additional layer(s) 82 provide the material for the formation of the inductor. The layer 82 has a very high conductivity. Suitable materials are gold, silver or platinum which, in addition to their high level of conductivity, are also characterized by their biocompatibility. When using less biocompatible electric conductors such as copper, a suitable plastic or ceramic coating may achieve the desired electrical insulation and biocompatibility . . . . According to FIG. 3, a coil with the material of FIG. 4a is formed as follows. The stent 1 consist of a two layered material that forms a honey-comb structure 101 and may, e.g., be cut from a pipe by means of laser cutting techniques. FIG. 3 shows the pipe folded apart. Thus, the left and the right side are identical. The conductive layer of the honey-comb structure is interrupted along the lines 9. For this purpose the conductive layer is cut during manufacture of the stent after the formation of the structure at the corresponding locations 91 by means of a chemical, physical or mechanical process. Such a location 91 where the conductive layer 82 disposed on the actual stent material is interrupted is schematically shown in FIG. 5.”

U.S. Pat. No. 6,280,385 also discloses that (in the paragraph beginning at line 55 of Column 9) “By the separation locations 91, the current path through the conductive material 82 is defined as it is indicated (by arrows 11) in FIG. 3. A coil arrangement 2 is created that forms the inductance of the stent 1. Conductive material for the coil function is selected in that the resistance through the conductor formed by the conductive material from one end to the other of the stent is lower than the default resistance through the stent material. The inductance 2 is formed automatically by the unfolding of the stent material during the application of the stent.”

One may also use a “three-layered material” to form the “inductor 22.” Thus, as is disclosed in the last paragraph of Column 9 of U.S. Pat. No. 6,280,385, “When using a three layered material according to FIG. 4b, the formation of an inductance takes place in a corresponding manner, whereby the layers of the conductive material are provided with separation locations for the formation of a current path. The use of two conductive layers has the advantage that the cross-section of the conductive track (land) is effectively doubled.” Referring to FIG. 1, the “inductor 22” may be coated with an insulating material 26 that preferably is biocompatible. Thus, as is disclosed in column 10 of U.S. Pat. No. 6,280,385, “In a further development of the exemplary embodiment of FIGS. 3 to 5, the conductive layer 82 is additionally coated with an insulating plastic such as a pyrolene in order to safely prevent current flow through the adjacent blood that would decrease the inductance of the coil. Pyrolenes are well suited since they are biocompatible and bond quite well with metal alloys. When coating the stent with pyrolenes after the manufacture process, the stent is held in a bath with pyrolenes or vaporized with pyrolenes.”

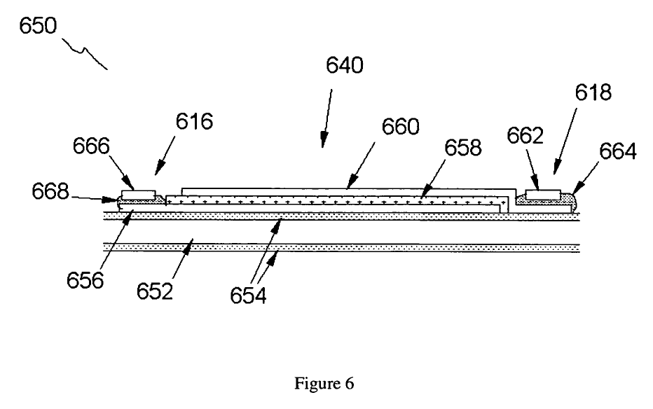

The inductor 22 may be provided by a helically shaped coil, as is disclosed in FIG. 6 of U.S. Pat. No. 6,280,385. As is disclosed in Column 10 of U.S. Pat. No. 6,280,385, “FIG. 6 depicts an alternative exemplary embodiment of a stent 1′, that forms an inductor 2′ and a capacitor 3′. The inductance here is provided in the form of a helix shaped coil 5 that is not formed by the skeleton of the stent itself, but is an additional wire woven into the stent skeleton 101. In this exemplary embodiment, the stent function and the coil function are separated. The coil 5 is again connected to a capacitor 3′ to form a resonance circuit that is either also a separate component or, alternatively, realized by adjacent coil turns or integrated surfaces of the stent. In applications of the stent, the coil 5, together with the stent material 101 having a smaller radius, is wound onto an application instrument such as a catheter and expands at the site of the application together with the stent material 101 to the desired diameter. Here the wire, that is, the coil 5, preferably is provided with a shape memory or the wire, that is, the coils 5, is/are preloaded on the application instrument.”

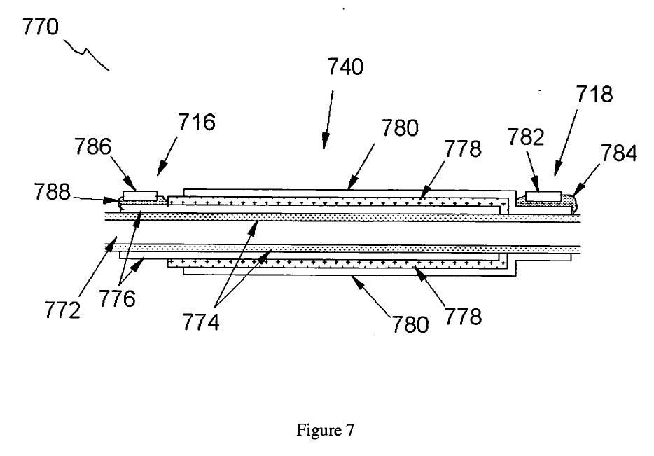

The inductor 22 may be, e.g., similar to the “inductor 2“” disclosed in FIG. 7 of U.S. Pat. No. 6,280,385. As is disclosed in the paragraph beginning at line 62 of Column 10 of such patent, “In the exemplary embodiment of FIG. 7, the inductor 2” of the stent is disclosed schematically. It can be formed either from the stent material (FIG. 3) or as an additional wire (FIG. 6). No individual capacitor is provided in this exemplified embodiment. Two loops 21, 22 of the inductance 2” actually form the capacitor whereby a dielectric 6 with a dielectric constant as high as possible is disposed between the loops 21, 22 for the increase of the capacitance.”

U.S. Pat. No. 6,280,385 also discloses that “In addition to the inductor 2”, an additional inductor 7 in the form of a coil pair 7 is provided, whereby its axis is perpendicular to the axis of the inductor 2”. The coil pair 7 is, for instance, formed by two spiral shaped coil arrangements that are integrated into the skeleton of the stent. This assures that in any arrangement of the stent in the tissue, one component is perpendicular to the direction of the field of the homogenous outer magnet. As an alternative to this arrangement, an additional inductance is provided vertical to the two depicted inductances. This assures an increased spin excitation in the observed region in every arrangement of the stent in the magnetic field.”

Referring again to FIG. 1, the inductor 22 may, e.g., be a receptor coil. As is disclosed, e.g., in Column 12 of the patent, “In a further development of the invention (not depicted), a catheter or balloon is equipped with a receptor coil apparatus. Instead of, or in addition to, an external receptor coil of the magnetic resonance system, the catheter or the balloon receives the signal amplified by the stent and transmits it extracorporeally. The catheter may be provided with the same or similar arrangement of inductor, capacitor and diodes and amplify the signals of the stent and transmit them by means of electrically conductive lands or by optical couplings and glass fibers extracorporeally to the tomograph. In comparison with the use of external receptor coils, this variant is characterized by improved signal detection. In a further development of the invention (not depicted), provisions made that the inductance of the stent itself is used as a receptor coil for the acquirement of magnetic resonance response signals, whereby the inductance is connected via cable connection to extracorporeal function components. It becomes possible to use the inductance of the resonance circuit complementary active for the imaging. Due to the necessity of a cable connection to extracorporeal function components, this in general will only be possible during the implantation of a stent.”

Referring again to FIG. 1, and in the preferred embodiment depicted therein, the inductor 22 preferably is coated with a biocompatible insulating material 26, regardless of whether the inductor 22 is a separate discrete component and/or an integral portion of the stent 12.

In one preferred embodiment, the material 26 is poly-p-xylylene. A description of poly-p-xylylene, processes for making it, and an apparatus in which deposition of such material may be effected may be found, e.g., in U.S. Pat. Nos. 3,246,627, 3,301,707, and 3,600,216, the entire disclosure of each of which is hereby incorporated by reference into this specification.

Reference also may be had, e.g., to pages 191-192 of J. R. Davis' “Handbook for Materials for Medical Devices,” ASM International, Materials Park, Ohio, 2003. As is disclosed in this work, “Parylene is a thin, vacuum-deposited polymer that is widely used for demanding medical coating applications . . . . It is based on a high-purity raw material called diparaxyylene, which is a white, crystalline powder. A vacuum and thermal process converts the powder to a polymer film, which is formed on substrates at room temperature . . . . Crystal-clear parylene film has very low thrombogenic properties and low potential for triggering an immune response.”

U.S. Pat. No. 5,380,320, the entire disclosure of which is hereby incorporated by reference into this specification, contains an excellent discussion of parylene film polymers. As is disclosed in Columns 2 and 3 of this patent, “Parylene is the generic name for thermoplastic film polymers based on para-xylylene and made by vapor phase polymerization. Parylene N coatings are produced by vaporizing a di(p-xylylene) dimer, pyrolyzing the vapor to produce p-xylylene free radicals, and condensing a polymer from the vapor onto a substrate that is maintained at a relatively low temperature, typically ambient or below ambient. Parylene C is derived from di(monochloro-p-xylylene) and parylene D is derived from di(dichloro-p-xylylene). Parylenes have previously been recognized as having generally good insulative, chemical resistance and moisture barrier properties. However, conventional parylene films do not generally adhere well to many substrate surfaces, particularly under wet conditions. Although these polymers are quite resistant to liquid water under most conditions, conventional parylene films are subject to penetration by water vapor, which can condense at the interface between the parylene film and the substrate, forming liquid water, which tends to delaminate the film from the substrate. In addition, conventional parylene films formed by vapor deposition are generally quite crystalline and are subject to cracking or flaking, which can expose the substrate below the film.”

U.S. Pat. No. 5,380,320 also discloses that “Parylene coatings have been used in the past in a wide variety of other fields, including the following . . . Christian et al. U.S. Pat. No. 5,174,295 Dec. 29, 1992 . . . Frachet et al. U.S. Pat. No. 5,144,952 Sept. 8, 1992 . . . Taylor et al. U.S. Pat. No. 5,067,491 Nov. 26, 1991 . . . Evans U.S. Pat. No. 4,950,365 Aug. 21, 1990 . . . Nichols et al. U.S. Pat. No. 4,921,723 May 1, 1990 . . . Bongianni U.S. Pat. No. 4,816,618 Mar. 28, 1989 . . . Bongianni U.S. Pat. No. 4,581,291 Apr. 8, 1986 . . . Japanese Patent 1297093 Nov. 30, 1989 . . . Christian et al. disclose a system for measuring blood flow using a Doppler crystal 251 having a thin protective coating of parylene (col. 19, line 57-col. 20, line 2) . . . Frachet et al. disclose a transcutaneous electrical connection device placed through the pinna of the ear or through the earlobe. The device includes at least one subcutaneous wire covered with an insulating sheath that is fixed to a metal ball positioned on the surface of the ear and covered with an insulating material on the part of its outer surface in contact with the ear. The insulating material and sheath are made of a bio-compatible material such as Teflon or parylene . . . Taylor et al. disclose a blood pressure-monitoring device for insertion into a patient's blood stream. The blood pressure-sensing element and catheter are conformably coated with a thin layer of parylene to insulate the device from the deleterious effects that blood components such as water and ions would otherwise have on various components of the device . . . . Evans discloses a process for coating a metal substrate by first applying a thin hard coated layer of titanium nitride, titanium carbide, or the like, and then a second coat of parylene . . . Nichols et al. disclose a process for applying an adherent electrically insulative moisture-resistant composite insulative coating to a substrate by glow discharge polymerization. Various parylenes are discussed as possible coating materials . . . . The Bongianni patents disclose a microminiature coaxial cable having a very thin ribbon strip conductor surrounded by a foamed dielectric or parylene. A thin coating of parylene is also applied to the outer conductor to prevent oxidation and inhibit mechanical abrasion . . . . Japanese Patent 1297093 discloses a pill cutter for woolen clothes in which a thin film of parylene (poly-para-xylylene) is formed on the surface of the cutting blade.”

U.S. Pat. No. 6,033,436, the entire disclosure of which is hereby incorporated by reference into this specification, discloses certain “stent coatings” that may be used as insulating material 26. Thus, and is disclosed in such patent (see Column 9), “It should be understood that all stent edges are preferably smooth and rounded to prevent thrombogenic processes and reduce the stimulation of intimal smooth muscle cell proliferation and potential restenosis. Accordingly, one embodiment of the invention, which is illustrated in FIG. 30, shows a ladder element 32 having rounded corners 60 and edges 70. Consequently, the implanted stent presents a substantially smooth intraluminal profile. Furthermore, the stent material may be coated with materials which either reduce acute thrombosis, improve long-term blood vessel patency, or address non-vascular issues. Coating materials that may be utilized to reduce acute thrombosis include: parylene; anticoagulants, such as heparin, hirudin, or warfarin; antiplatelet agents, such as ticlopidine, dipyridamole, or GPIIb/IIIa receptor blockers; thromboxane inhibitors; serotonin antagonists; prostanoids; calcium channel blockers; modulators of cell proliferation and migration (e.g. PDGF antagonists, ACE inhibitors, angiopeptin, enoxaparin, colchicine) and inflammation (steroids, non-steroidal anti-inflammatory drugs). Coating materials which may be used to improve long-term (longer than 48 hours) blood vessel patency include: angiogenic drugs such as, Vascular Endothelial Growth Factor (VEGF), adenovirus, enzymes, sterol, hydroxylase, and antisense technology; drugs which provide protection on consequences of ischemia; lipid lowering agents, such as fish oils, HMG, Co-A reductase inhibitors; and others. Finally, drugs that address nonvascular issues such as ibutilide fumarate (fibrillation/flutter), adenylcyclase (contractility), and others, may be applied as stent coatings.” These coatings are also described in claims 13 and 14 of the patent. Claim 13 of U.S. Pat. No. 6,033,436 describes “13. The expandable stent of claim 1, wherein the ladder elements are treated with a coating agent which reduces acute thrombosis, improves long-term blood vessel patency, or addresses non-vascular issues.” Claim 14 of such patent describes “14. The expandable stent of claim 13, wherein the coating agent is selected from the group consisting of parylene, heparin, hirudin, warfarin, ticlopidine, dipyridamole, GPIIb/IIIa receptor blockers, thromboxane inhibitors, serotonin antagonists, prostanoids, calcium channel blockers, PDGF antagonists, ACE inhibitors, angiopeptin, enoxaparin, colchicine, steroids, non-steroidal anti-inflammatory drugs, VEGF, adenovirus, enzymes, sterol, hydroxylase, antisense sequences, fish oils, HMG, Co-A reductase inhibitors, ibutilide fumarate, and adenylcyclase.”

Referring again to FIG. 1, and in one preferred embodiment, the inductor 22 is coated with a material 26 comprised of blood compatible material that does not cause thrombogenic behavior. These blood compatible coatings are well known to those skilled in the art. Thus, e.g., as is disclosed on pages 192-193 of J. R. Davis' Handbook of Materials for Medical Devices, “Improved compatibility with blood is a desired feature for a variety of medical devices that must contact blood during clinical use. The materials used for manufacture of medical devices are not inherently compatible with blood and its components. The response of blood to a foreign material can be aggressive, resulting in surface-induced thrombus (clot) formation, which can impair or disable the function of the device and, most importantly, threaten the patient's health. Even with the use of systemic anticoagulants, the functioning of devices such as cardiopulomonary bypass circuits, hemodialyzers, ventricular-assist devices, and stents has been associated with thrombus formation, platelet and leucocyte activation, and other complications related to the deleterious effects of blood/material interactions.”

One may use one or more of the blood compatible coatings disclosed in prior art patents. Thus, by way of illustration and not limitation, one may use the copolymeric material disclosed in U.S. Pat. No. 4,083,832, the entire disclosure of which is hereby incporated by reference into this specification. This patent claims “1. A blood-compatible substantially non-thrombogenic shaped article of water-insoluble solid copolymer of 10 to 60 mole percent styrene and 90 to 40 mole percent of at least one copolymerizable acyclic aliphatic comonomer of the formula CH2═C(R′)—X wherein R′ is H or CH3, X is —CN, —COOH or —COOR′ and R′ is unsubstituted lower alkyl of 1-6 carbon atoms, said article having a ciliate hydrophilic surface containing 20 to 400 milliequivalents of sulfonic acid groups per square meter, said sulfonic acid groups being in the para-position of the aromatic nucleus of said styrene, and a electronegativity measured on detached portions of said ciliate hydrophilic surface as a zeta potential of from −5 to −70 millivolts.” At Columns 1-2 of this patent, the copolymeric materials are referred to. It is disclosed that: “It has now been found that a particular class of surface-sulfonated styrene-aliphatic vinyl compounds provides vessels and containers for handling blood in accordance with the objects of the invention. These materials are related to but not identical with the slippery polymers disclosed and claimed in Campbell et al., U.S. Ser. No. 336,244, filed Feb. 27, 1973. The present materials differ from the broad class of sulfonated polymers therein disclosed and claimed in that lower proportions of the aromatic constituent, styrene, are used together with lower proportions of an aliphatic vinyl compound comonomer. Furthermore, as will become evident hereinafter, particular properties are needed for the sulfonated polymer to have enhanced non-thrombogenicity. It is found that this class of sulfonated polymers provides a surface region which is hydrophilic and imbibes and holds water, which is securely bonded to and an integral part of the hydrophobic polymer substrate, but which differs chemically and morphologically from the typical, uniformly crosslinked hydrogel described above. The novel surface region is obtained when certain styrene aliphatic vinyl copolymers are sulfonated so that hydrophilic groups are attached to the polymer chains in a graded manner, the outermost polymer chains receiving the largest number of hydrophilic groups per given unit of chain length, the number of hydrophilic groups per unit of chain length, the number of hydrophilic groups per unit of chain length diminishing in the direction of the interior until the composition of the unmodified interior is reached. Articles of styrene-aliphatic vinyl copolymers are thus provided with a hydrophilic skin or zone on whatever surfaces are treated. Because of the gradation in hydrophilicity in this zone, the outermost portions of this hydrophilic zone becomes highly hydrated and swollen in contact with aqueous media, but swelling diminishes progressively in the direction of the interior of the solid and the interior portion of the treated zone will not become hydrated and consequently remains firmly fixed in the hydrophobic solid. Portions of the skin can be removed by scraping with a spatula so that cataphoretic, i.e., electrophoretic, properties can be determined.”

U.S. Pat. No. 4,083,832 also discloses that “A distinctive feature of the hydrophilic skin or zone is that when totally immersed in aqueous media its outermost, most highly swollen portion breaks up into microscopic and submicroscopic strands or cilia which are fixed at their interior ends and project into the aqueous phase. This is termed a ciliate surface. These strands are highly flexible. The outermost region of the hydrophilic zone approaches a condition of high dilution in the aqueous medium. These materials having hydrophilic zones or skins have been found to be outstanding in their ability to function in contact with blood, demonstrating a high degree of non-thrombogenicity and being non-adherent and non-damaging to blood cellular elements.”

By way of further illustration, one may use one or more of the “blood compatible polymers” described in U.S. Pat. No. 4,210,529, the entire disclosure of which is hereby incorporated by reference into this specification. Claim 6 of this patent describes “6. A laminate comprising a coating on a gas-permeable, porous polyolefin substrate, said coating comprising a fluoroacylated ethyl cellulose derivative having about 4.4 to about 5.5 ethoxide groups per disaccharide unit, whereby essentially all of the pendant methylol groups and more than about 50 mole % of the ring-substituted hydroxyls of said ethyl cellulose derivative are etherified, said ethyl cellulose derivative containing about 0.5 to about 1.6 ring-substituted —OCO(CF2)m CF3 groups per disaccharide unit, wherein m is a number ranging from 1 to 6, said ethyl cellulose derivative containing, at most, trace amounts of residual hydroxyl groups as determined by infrared spectroscopic analysis and having a calculated chemically combined fluorine content of more than 10% by weight.”

The inducement of blood clots by “foreign substances” is described at Columns 1-2 of U.S. Pat. No. 4,210,529, wherein it is disclosed that “It has long been recognized that an enormous variety of organic and inorganic foreign substances, when brought into contact with the blood of a living animal, will stimulate the formation of blood clots. However, it has only been relatively recently that the clotting or thrombogenic effect of foreign substances has been investigated very effectively from a theoretical standpoint. For example, the electronegative character of the endothelial wall of the circulatory system was not fully recognized until the 1950's. The study of interactions between plasma proteins and/or cellular elements of blood (e.g. platelets) and foreign substances is still far from comprehensive. A good summary of the present understanding of these phenomena is contained in Chapter III of Blood Compatible Synthetic Polymers by S. D. Bruck, published by C. C. Thomas, Springfield, Ill., 1974. Dr. Bruck also summarizes the still largely empirical approaches toward the syntheses of relatively blood-compatible (relatively non-thrombogenic) polymers.”

U.S. Pat. No. 4,210,529 also discloses that “As a general rule, the properties desired in a blood oxygenator membrane include: good gas permeability (at least with respect to gaseous oxygen and carbon dioxide); chemical stability (particularly at the blood pH of 7.4 and at temperatures within the range of 20°-40° C., but preferably also at other pH's and temperatures used in sterilization (e.g. 100° C.); blood compatibility or substantially non-thrombogenic behavior in blood-containing environments; sufficiently hydrophobic character to serve as a water vapor barrier; ease in manufacture (e.g. sufficient solubility to permit solvent casting or the like); non-toxicity; relative inertness to body fluids; and mechanical strength and handling properties adequate for facilitating the assembly and use of the blood oxygenation devices. Unfortunately, it is difficult to combine non-thrombogenic behavior with other properties which are necessary or desirable in blood oxygenators. For example, attempts have been made to improve the blood compatibility of polydimethylsiloxane. This class of polymers, except for its adverse tendencies with respect to various blood components (absorption of lipids, promotion of platelet adhesion, and the like), can have some physical and chemical properties which are very useful in blood oxygenation devices, including good gas permeability. It has been suggested that side chains with negative polarity attached to the siloxane polymer backbone would improve the blood compatibility of this class of polymers, and partially fluorinated polysiloxanes have been synthesized and tested for both gas permeability and blood compatibility. The introduction of the fluorine-containing side chains resulted in “lowered permeability toward oxygen and carbon dioxide in comparison to polydimethylsiloxane and mixtures of polydimethylsiloxane and fluorinated polysiloxanes. Blood compatibility data for the fluorosiloxane elastomers suggest that the 65/35 mole percent blend of fluorosiloxane/dimethylsiloxane performs somewhat better than the fluorosiloxane homopolymer, despite the larger number of fluorine groups present in the latter” (S. D. Bruck, op. cit., page 76).”

U.S. Pat. No. 4,210,529 also discloses that “To further illustrate the problems of synthesizing or discovering the ideal blood oxygenation membrane, perfluoresters of poly(ethylene-vinyl alcohol) copolymers have been made and found to be hydrolytically unstable under room temperature and ordinary atmospheric moisture conditions. These esterified copolymers are made by reacting the pendant hydroxyls of the vinyl alcohol units with perfluorobutyric acid chloride. Hydrolysis at room temperature in the presence of moisture at a pH of 7 regenerates free hydroxyl. For a report on the thrombo resistance of perfluoroacetate esters of poly(ethylene-vinyl alcohol), see Gott, NTIS annual report PB 186,551 (August, 1969), p. 65 et seq.”

U.S. Pat. No. 4,210,529 also discloses that “Fluorinated polyacrylate esters have also been investigated, in this case from the standpoint of gas permeability. Presently available data indicate that fluorination has no significant favorable effect upon the gas permeability of these acrylate polymers. As illustrative of the state of the fluorinated film-forming polymer art, see British Pat. No. 1,120,373 (ICI, Ltd.), published Jul. 17, 1968.”

By way of yet further illustration, one may use the hemocompatible plastic material disclosed in U.S. Pat. No. 4,286,597, the entire disclosure of which is hereby incorporated by reference into this specification. Claim 1 of this patent describes “1. The method of storing blood which comprises placing said blood for a period of days into a flexible, hemocompatible, sterilizable, chlorine-free plastic material which contains sufficient dioctyladipate to cause a reduced plasma hemoglobin content of blood stored in contact therewith for 21 days, when compared with blood stored in contact”

By way of yet further illustration, one may use the “biological” described in U.S. Pat. No. 4,820,302, the entire disclosure of which is hereby incorporated by reference into this specfification. This patent claims “1. An implantable breast prosthesis comprising: a three dimensional member having an outer surface at least a portion of which is adapted to be in contact with tissue and the like in a host recipient, at least said portion of said member having functional groups extending therefrom, said functional group being selected from the group consisting of primary and secondary amino groups, said amino groups having attached thereto a reactive group selected from the group consisting of aldehyde and aryl-halide groups, a biological having functional groups selected from the group consisting of primary and secondary amino groups and hydroxyl groups being coupled to said portion of said member through reaction between the functional groups of said biological and the aldehyde or aryl-halide groups attached to said primary or secondary amino groups which extend from said portion of said member, and said biological operating to provide bio- and blood compatible qualities to said surface portion.”

U.S. Pat. No. 4,820,302 presents an excellent discussion of “blood compatible materials.” Thus, and as is disclosed in such patent, “There are many instances in medicine in which there is a need for a bio- and blood-compatible material for human and animal use and for use in equipment contacted by biologicals or blood, e.g., tubing, containers, valves, etc. For example, in extra-corporeal circulation of blood, i.e., heart or artificial kidney there is a tendency for blood to coagulate on contact with a “foreign surface”, see, for example, U.S. Pat. Nos. 3,642,123 and 3,810,781. Also, products such as heart valves, materials used in coronary and vascular grafts, and catheters, oxygenator tubing and connectors tend to cause thrombosis of the blood. ressings and surgical dressings should be bio- and blood compatible. In the case of such dressings, an area in which the present invention finds particular utility, there are additional requirements because of the use of the materials.”

U.S. Pat. No. 4,820,302 also discloses that “Accordingly, a wide variety of dressings, characterized as biological and synthetic, have been used in the treatment of burn wounds. Biological dressings include any dressing that has one or more biological components, i.e., protein, carbohydrates, lipids and the like. Presently, homograft and porcine xenograft skin are dressings currently used to maintain the granulation bed of burn tissue, the amount of available skin (autograft) is limited and temporary dressings are required for long periods of time to maintain the granulation bed. Homografts (cadaver skin) is the current dressing of choice, when available. Unfortunately, homograft has a limited shelf life and is relatively expensive, i.e., $85.00 to $90.00 per square foot. Human amniotic membrane has also been used but is less desirable than cadaver skin. Lack of availability and short shelf life are also drawbacks.”

U.S. Pat. No. 4,820,302 also discloses that “Xenograft (porcine) skin is commercially available but is considerably less effective than homografts and autografts. Short shelf life, sterility and limited application are known disadvantages of this material, in addition to an antigenicity problem.”

U.S. Pat. No. 4,820,302 also discloses that “In addition to the materials previously mentioned, various forms of collagen have been used in the treatment of burns, see U.S. Pat. No. 3,491,760 which describes a “skin” made from two different tanned collagen gel layers. U.S. Pat. No. 3,471,958 describes a surgical dressing made up of a mat of freeze dried microcrystalline collagen, while British Pat. No. 1,195,062 describes the use of microcrystalline colloidal dispersions and gels of collagen to produce films which are then applied to various fibers such as polyurethane.”

U.S. Pat. No. 4,820,302 also discloses that “A “biolization” process for improving the blood and biocompatibility of prosthetic devices has been described by Kambic, et al and others, see Trans. 3rd Annual Meeting Society for Biomaterials, Vol. 1, p. 42, 1977. Their methods involve deposition of gelatin into a rough textured rubber with subsequent cross-linking and stabilization of the gelatin with 0.45% gluteraldelyde.”

U.S. Pat. No. 4,820,302 also discloses that “There are numerous references in the literature to various other materials used in burn treatment. For example, collagen membranes have been fabricated from suspensions of bovine skin and evaluated in a rat animal model. The adherence of this material was superior to auto- homo- and xenografts on full and split thickness wounds but inferior to auto- and homografts on granulating wounds, see Tavis et al, J. Biomed. Mater. Res. 9, 285 (1975) and Tavis et al, Surg. Forum 25, 39 (1974).”

U.S. Pat. No. 4,820,302 also discloses that “McKnight et al, developed a laminate of collagen foam with a thin polyurethane sheet, see U.S. Pat. No. 3,800,792. Film prepared from reconstituted collagen has also been used, Tavis et al, supra, and a commercially grade of such material is available from Tec-Pak Inc. Gourlay et al, Trans, Amer, Soc, Art, Int. Organs 21, 28 (1975) have reported the use of a silicone collagen composition, collagen sponge, and non-woven fiber mats.”

U.S. Pat. No. 4,820,302 also discloses that “In addition to the above, U.S. Pat. No. 3,846,353 describes the processing of silicone rubber with a primary or secondary amine, see also Canadian Pat. No. 774,529 which mentions ionic bonding of heparin on various prosthesis. In addition to the above, there is considerable literature relating to the use of silicone rubber membranes Medical Instrumentation, Vol. 7, U.S. Pat. No. 4,268,275 September-October 1973; fabric reinforced silicone membranes, Medical Instrumentation, Vol. 9, U.S. Pat. No. 3,124,128, May-June 1975. U.S. Pat. No. 3,267,727 also describes the formation of ultra thin polymer membranes.”

U.S. Pat. No. 4,820,302 also discloses that “It is also known that various materials may be heparinized, in order to impart a non-thrombogenic character to the surface of a material, see for example U.S. Pat. Nos. 3,634,123; 3,810,781; 3,826,678; and 3,846,353, and Canadian Pat. No. 774,529, supra.”

U.S. Pat. No. 4,820,302 also discloses that “In addition to the above, there is a significant body of art dealing with mammary prostheses formed of a particular material, see for example Calnan et al, Brit. J. Plast. Surg., 24(2), pp. 113-124(1971); Walz, Med. Welt. 30(43), pp. 1587-94 (Oct. 26, 1979), and Bassler, Zeitschrift fur Plastische Chirurgie 3 (2), pp. 65-87 (July, 1979). Also present in the art are disclosures of bio- and blood compatible substrates through the use of biofunctional surfaces. For example, Ratner et al, J. Biomed. Mater. Res., Vol. 9, pp. 407-422 (1975) describes radiation-grafted polymers on silicone rubber sheets. U.S. Pat. Nos. 3,826,678 (Hoffman et al issued Jul. 30, 1974) and U.S. Pat. No. 3,808,113 (Okamura et al issued Apr. 30, 1974) describes the use of serum albumin and heparin as a biological coating, and collagen cross-linked by radiation. Collagen muco-polysaccharide composites are described by Yannas et al in U.S. Pat. No. 4,208,954 issued on July 28, 1981 while Yannas et al U.S. Pat. No. 4,060,081 of Nov. 29, 1977 describes a multi-layered membrane for control of moisture transport in which cross-linked collagen and muco-polysaccharide is said to preclude immune response. Eriksson et al in U.S. Pat. No. 4,118,485 of Oct. 3, 1978 describes a non-thrombogenic surface using heparin.”

One may use the process described in U.S. Pat. No. 4,828,561 to prepare “blood compatib 1. Method for applying a blood-compatible coating to a polyether-urethane moulded article, characterized in that a layer of polyethylene oxide with a weight average molecular weight in the range of 1,500-1,500,000 is applied to the polyether-urethane moulded article using heat treatment or irradiation and the polyethylene oxide layer applied is then linked to the polyethylene-urethane moulded articlele coatings.” Claim 1 of this patent, the entire disclosure of which is hereby incorporated by reference into this specification, describes “A method for imparting bio and blood compatible characteristics to a substrate wherein said substrate is a material which includes at least a surface portion which is a silicone polymer material, comprising the steps of: treating at least a portion of the surface of said substrate to provide functional reactive sites selected from the group consisting of primary and secondary arnine functional sites coupled directly to at least the silicone polymer material; activating said functional reactive sites with a material selected from the group consisting of an aryl halide and a dialdehyde to provide active connecting groups selected from the group consisting of aldehyde and halide connecting groups; and coupling to said connecting groups a biological having a functional group selected from the group consisting of hydroxyl, primary amine, and secondary amine functional groups, for reaction with said connecting groups to form a biological covalently bound to at least a portion of said substrate to impart thereto bio and blood compatible characeristics to at least a portion of the surface of said substrate.”

By way of yet further illustration, one may use the blood-compatible coating disclosed in U.S. Pat. No. 4,965,112, the entire disclosure of which is hereby incorporated by reference into this specification. Claim 1 of this patent describes “Method for applying a blood-compatible coating to a polyether-urethane moulded article, characterized in that a layer of polyethylene oxide with a weight average molecular weight in the range of 1,500-1,500,000 is applied to the polyether-urethane moulded article using heat treatment or irradiation and the polyethylene oxide layer applied is then linked to the polyethylene-urethane moulded article.”

U.S. Pat. No. 4,965,112 has an excellent discussion of the phenomena that give rise to thrombi. As is disclosed in Columns 1-2 of this patent, “As is generally known, polyether-urethanes have found application in numerous biomedical fields by virtue of their good physical and mechanical properties and also their relatively good compatibility with blood. However, it has been found that for the vast majority of these polyether-urethane elastomers this compatibility with blood still leaves something to be desired for certain applications, in particular in the case of long-term contact with blood or body tissue. It is in particular the surface, or more accurately the surface characteristics, of the moulded articles which play a significant, if not decisive, role here. Specifically, the surface of exogenic materials must possess an adequate resistance to blood coagulation, blood platelet adhesion etc. on contact with body tissues and blood. Thrombogenesis, embolization and the like are, therefore, frequently the cause which makes the application of biomedical moulded articles doomed to failure. More particularly, the use of the majority of non-physiological biomaterials such as polyether-urethanes after contact with, for example, blood gives rise within a very short time to a thin protein-like layer on these materials, which layer is rich in fibrinogen, fibronectin and gamma-globulin. By reason of the circulation of the blood, further protein components will adhere firmly to this initially thin layer, so that phenomena can arise which lead to activation of the defence mechanism, such as coagulation, blood platelet adhesion, adhesion of white blood cells and the like.”

U.S. Pat. No. 4,965,112 also discloses that “In view of the disadvantages, described above, of the use of synthetic biomedical materials, methods for eliminating or greatly reducing the undesired phenomena associated with the use of moulded articles produced from these biomedical materials has been diligently sought. One of the methods was directed towards the modification of the surface of biomedical materials, polyether-urethanes in the present case, to attempt to prevent the endogenic protein adhesion and agglomeration. The process known from EP-A-0,061,312 for the application of covalently bonded aliphatic chains with 14-30 carbon atoms to the substrate surface, for example of polyurethane, is mentioned as one of the methods. Preferably, n-octadecyl groups are attached to the polymer substrate surface. Coated substrates of this type possess selective and apparently reversible bonding sites for albumin, so that the adherence of thrombogenic proteins is largely prevented. Five methods for the covalent bonding of the long aliphatic chains to the substrate surface are described in this EP-A-0,061,312, a proton-removing base such as sodium ethoxide (NaOEt), sodium t-butyrate (NaO.t.Bu), potassium hydride or sodium hydride and methyl magnesium bromide always having to be used.”

U.S. Pat. No. 4,965,112 also discloses that “In view of the specific but somewhat laborious methods of preparation of polymer substrates coated with alkyl groups having 14-30 carbon atoms known from EP-A-0,061,312, the Applicant has sought for a method which is simple in respect of technique for immobilizing a synthetic polymer layer on a polyether-urethane moulded article which possesses an outstanding compatibility with blood. It has been found that the aim of the invention can be achieved when a layer of polyethylene oxide with a Mw in the range of 1,500-1,500,000, preferably 100,000-300,000, is applied directly to a polyether-urethane moulded article and the polyethylene oxide layer applied is then linked to the polyether-urethane moulded article. Surprisingly, very simple techniques, such as a heat treatment or irradiation with UV light, can be used for this linking.”

U.S. Pat. No. 4,965,112 also discloses that “More particularly, the thermal linking according to the invention is carried out at a temperature in the range of 80-180° C., preferably 100-150° C. Advantageously, the thermal linking is carried out in the presence of an organic peroxide which can be used at this temperature, for example of the formula R—O—O—R′ in which R and R′ independently of one another represent a straight-chain or branched alkyl group with 4-10 carbon atoms, a cycloalkyl group with 5-8 carbon atoms or an aralkyl group with 6-10 carbon atoms. Dicumyl peroxide is mentioned as an example of a peroxide which can be used.”

By way of yet further illustration, one may use the “blood compatibile medical material” disclosed in U.S. Pat. No. 5,336,698, the entire disclosure of which is hereby incorporated by reference into this specification. This patent claims (in claim 1 thereof) “1. A blood-compatible medical material comprising: a base material (A) comprising at least one member selected from the group consisting of cellulose, polyvinyl alcohol, polyvinyl acetate, copolymers of ethylenevinyl alcohol, copolymers of ethylenevinyl acetate. poly(meth)acrylic acid, chitin, chitosan, collagen, and polyacrylamide; a copolymer (B) covalently bonded to said base material having as a component at least one member selected from the group consisting of glycidyl (meth)acrylate, alkyl(meth)acrylate, glycidyl (meth)acrylate-(meth)acrylic acid, (meth)acryloxy alkyl alkoxy silane, (meth)acrylic and alkyl(meth)acrylic acid; and a component selected from the group consisting of a fatty acid ester of a fatty acid and an alkylene glycol or an amide of fatty acid and alkylene diamine, covalently bonded to said copolymer (B).”

By way of yet further illustration, one may use the “composition compatible with blood” disclosed in U.S. Pat. No. 5,541,305, the entire disclosure of which is hereby incorporated by reference into this specification. Claim 1 of this patent describes “A composition compatible with blood prepared by ion exchange complexation of a polymer having quaternary ammonium groups with an alkali metal salt of a polyanion selected from the group consisting of heparin, chondroitin sulfate, dextran sulfate, and polyvinyl alcohol sulfate, wherein said polymer having quaternary ammonium groups is prepared by quaternizing a polymer containing tertiary amino groups with a quaternizing agent, and wherein the equivalent ratio, M/S, of alkali metal atoms (M) to sulfur atoms (S) in the composition is 0.4 or less.”

One may also use the “blood compatible surface layer” disclosed in U.S. Pat. No. 5,728,437, the entire disclosure of which is hereby incorporated by reference into this specification. Claim 1 of this patent describes “Article exhibiting at least one hydrophobic surface of glass, metal or a hydrophobic polymer coated with a blood compatible surface layer, wherein the blood compatible surface layer consists of an adsorbed ethyl-hydroxyethyl-cellulose having a flocculation temperature of about 35°-40° C.”

At Columns 1-2 of U.S. Pat. No. 5,728,437, a description of the “prior art” is presented. It is disclosed that Prior art technique to provide articles useful in medicine with a blood-compatible surface layer often comprises an alteration in the surface energy of the material. An improvement in the properties of various materials has been obtained by modifying the surface layers either to a more hydrophobic character or to a more hydrophilic character. Hydrophobization of the surface layer, for instance by the methylization of a glass surface, results in a decrease in the effectiveness of the surface activated coagulation system of the blood. However, proteins such as fibrinogen are bound comparatively firmly to such surface and to this protein layer certain cells, the thrombocytes, can be bound and activated whereafter coagulation is started even though it proceeds slowly. Hydrophilic surfaces, e.g. hydrolysed nylon or oxidized aluminium, have presented reduced binding of cells but the surface activated coagulation system is not prevented at these surfaces. The use of these surfaces in contact with blood thus implies the addition of anti-coagulants, for instance heparin to the blood.”

U.S. Pat. No. 5,728,437 also discloses that “Another prior art surface treatment technique for preventing coagulation comprises binding of anticoagulants into the surface layer. Heparin has primarily been used with this technique. Heparin is a hexoseamine-hexuronic acid polysaccharide which is sulphatized and has acid properties, i.e. heparin is an organic acid. According to DE-A-21 54 542 articles of an organic thermoplastic resin is first impregnated with an amino-silane coupling agent and the article thus treated is then reacted with an acid solution of heparin salt to the binding of heparin in the surface layer by means of ionic bonds. Surfaces thus treated with heparin have proved to reduce the coagulation reaction. A considerable disadvantage of these surfaces, however, is that the heparin treatment does not prevent the adherence of thrombocytes, which is a great problem in, for instance, heart-lung machines.”

U.S. Pat. No. 5,728,437 also discloses that “On the 10th Annual Meeting of the Society for Biomaterials (Washington D.C. Apr. 27, 1984) was described that polyethylenglycol surfaces on quartz minimize protein adsorption. Procedures for covalent binding of polyethylenglycol to surfaces have previously been described, e.g. in WO86/02087. Polyion complexes formed between a cationic and an anionic cellulose derivative have also been found to have good blood-compatibilities (Ito, H. et. al., J. Appl. Polym. Sci., Vol. 32 (1986) 3413). Methods of covalent binding of water-soluble polymers to surfaces have also been described, e.g. in EP 166 998.”

U.S. Pat. No. 5,728,437 also discloses that “It is known that water-binding gels, for instance polyhydroxyalkylmethacrylate, reduce the adsorption of proteins and present a low adhesiveness to cells (Hoffman et al., Ann. N.Y. Acad. Sci., Vol. 283 (1977) 372). These properties are considered to be due to the fact that gels containing water give a low surface energy in the interface to the blood. The prior art technique for manufacturing of water-binding gels, however, is impaired by disadvantages such as complicated preparation technique and incomplete polymerization, which results in leakage of toxic monomers. A gel-like mixture of saccharose and glucose included in a matrix of the polysaccharide dextran or dextrin is used in accordance with previously known technique as a robe for the connection of blood-vessels. This mixture should have the effect that no toxicity to the patient occurs that the implantate is dissolved in the blood after some time. It is known that the neutral polysaccharide dextran is miscible with blood without provoking any coagulation reaction. Dextran has been used as a surface coating on glass, aluminium and silicon rubber, and has been shown to reduce blood coagulation during blood contact with these surfaces as described in WO83/03977. The adhesion of blood components to surfaces in contact with blood could be decreased by preadsorption of albumin to hydrophobic surfaces (Mosher, D. F, in: Interaction of blood with natural and artificial surfaces, Ed. Salzman, E. W., Dekker Inc 1981). The adsorbed albumin does not form a stable coating, but is desorbed during contact with blood and coagulation is induced although at a lower rate.”

One may also use the “blood compatible antimicrobial surface” disclosed in U.S. Pat. No. 6,022,553, the entire disclosure of which is hereby incorporated by reference into this specification. A process for preparing such a surface is described in claim 1 of such patent, which discloses “. A method for forming a bacteria-repelling and blood-compatible modified surface on a substrate, comprising the sequential steps of: a. activating the surface of a substrate; b. grafting the resulting activated surface of the substrate with a hydrophilic monomer, and c. subjecting the resulting grafted substrate to an SO2 plasma treatment, whereby bacterial adhesion and blood platelet adhesion to said modified surface after exposure to said plasma treatment is less than prior to said plasma treatment.” In Columns 1-2 of this patent, it is disclosed that “In addition to being susceptible to microbial contamination, medical articles used as implants can cause dangerous blood clots. The clots are started when blood cells and other blood particles, such as thrombocytes, adhere to the surface of the implanted device. While certain disinfectants (e.g. benzalkonium chloride/heparin) have been shown to reduce the incidence of clotting, they have poor adherence to the underlying substrate, and quickly dissolve off the surface of the implanted device.”

U.S. Pat. No. 6,022,553 also discloses that “It has been reported that membranes treated with a low pressure plasma are less likely to cause blood clotting, i.e. be thrombogenic, than similar, untreated membranes (International Patent Application WO 94/17904. In the description of the treatment method, SO2 was mentioned as a suitable plasma forming gas. There have been additional reports on using SO2 as a plasma forming gas in the plasma treatment of LDPE tubes (J. C. Lin et al., Biomaterials 16 (1995), 1017-1023. The authors reported that the surfaces modified by SO2 plasma treatment were strongly hydrophilic, and more thrombogenic than untreated surfaces. They attributed this result to the addition of polar sulfonate groups, created by the SO2 plasma treatment, to the already hydrophilic surface of the LOPE tubes.”

By way of yet further illustration, one may the “protective stent coating” disclosed in U.S. Pat. No. 6,174,329, the entire disclosure of which is hereby incorporated by reference into this specification. Claim 6 describes a “blood compatible protective layer” as being “. . . formed from a polymeric material selected from the group consisting of Parylast®, Parylene, polymethylene, polycarbonate-urethane copolymer, silicone rubber, hydrogels, polyvinyl alcohol, polyvinyl acetate, polycapralactone, urethanes, and PHEMA-Acrylic.”

In the first two columns of U.S. Pat. No. 6,174,329, a discussion is presented of a stent comprised of two dissimilar metals in direct contact. It is disclosed that “body that is substantially radiolucent and is formed from, for example, a stainless steel alloy. In order to increase the radiopacity of the stent, without the disadvantages of thicker wires, the stent, or a portion thereof, is coated with a thin radiopaque layer of material having high atomic weight, high density, sufficient surface area and sufficient thickness. With such a coating, the stent is sufficiently radiopaque to be seen with fluoroscopy, yet not so bright as to obstruct the radiopaque dye. This radiopaque layer covers at least a portion of the stent and can be formed from gold, tantalum, platinum, bismuth, iridium, zirconium, iodine, titanium, barium, silver, tin, alloys of these metals, or similar materials.”

U.S. Pat. No. 6,174,329 also discloses that “The radiopaque layer is thin, in one preferred embodiment it is about 1.0 to 50 microns thick. Since the layer is so thin, it is subject to scratching or flaking when the stent is being delivered intraluminally. Accordingly, it is an object of the invention to protect the stent and particularly the radiopaque layer with a more durable protective layer that is resistant to scratching and general mishandling. Whenever two dissimilar metals are in direct contact, such as a stainless steel stent at least partly covered with a gold radiopaque layer, there is the potential to create the electrochemical reaction that causes galvanic corrosion. The by-product of corrosion (i.e., rust) will not be biocompatible or blood compatible, may cause a toxic response, and may adversely affect adhesion of the radiopaque material. Corrosion will occur if gold and another metal, like stainless steel, are in contact with the same bodily fluid (electrolyte). If the gold coating has any pinhole or has flaked or scratched off the surface, the underlying stainless steel will be exposed to the same fluid. Therefore, a galvanic reaction (battery effect) will occur. The use of a single protective coating covering the entire surface prevents this reaction. This is especially pertinent when the radiopaque layer partially covers the stainless steel stent. The protective layer of the present invention also prevents galvanic corrosion so that the stent is biocompatible.”

By way of yet further illustration, one may use the blood-compatible composition described in U.S. Pat. No. 6,200,588, the entire disclosure of which is hereby incorporated by reference into this specification. This patent claims “1. A blood-compatible composition comprising an ionic complex comprising at least two organic cationic compounds and heparin or a heparin derivative, wherein said at least two organic cationic compounds comprise at least the following two compounds (a) and (b): (a) a compound selected from a group consisting of an ammonium compound and a phosphonium compound, both having four aliphatic alkyl groups, wherein two of the four aliphatic alkyl groups are methyl and the other two are long chain aliphatic alkyl groups having 12 carbon atoms, and (b) a compound selected from the group consisting of an ammonium compound and a phosphonium compound, both having four aliphatic alkyl groups, wherein said compounds have at least two alkyl groups having not less than 10 carbon atoms each and wherein the four aliphatic alkyl groups have 30 to 38 carbon atoms in total.

At Columns 1-2 of U.S. Pat. No. 6,200,588, antithrombogenicity is discussed. It is disclosed that “Along with the progress of medicine, more medical devices made from a polymer material have been widely used, and highly advanced medical devices such as assistant circulation devices (e.g., artificial heart, artificial kidney, pump-oxygenator, intra-aortic balloon pumping and the like), catheters for various diagnoses and therapies, synthetic vascular prosthesis and the like have been put to practical use. However, most of these medical devices are made from polymer materials developed for industrial use without modification, and they require a combined use of an anticoagulant when in use, that prevents coagulation of blood on contact with the medical devices.”

U.S. Pat. No. 6,200,588 also discloses that “However, anticoagulants not only prevent coagulation on the surface of a medical device but also deprive systemic hemostatic function. The use, therefore, is associated with the risk of causing complications such as hemorrhage at the site of insertion or use of medical device, at an operative wound and, in a serious case, from a cerebral vessel. Thus, in an attempt to prevent the above-mentioned complications, methods have been studied that involve imparting antithrombogenicity to a medical device, thereby to reduce administration of anticoaglant.”

U.S. Pat. No. 6,200,588 also discloses that “As a method for imparting antithrombogenicity to a medical device, there have been practiced (A) a method comprising mixing highly fine particles of a polymer material and an anticoagulant substance (e.g., heparin), dispersing the mixture in a solvent and applying the resulting dispersion onto a medical device, (B) a method comprising introducing cation groups such as quaternary ammonium salts into a polymer, dissolving the cation group-containing polymer in a solvent, applying the solution onto a medical device and bringing an aqueous solution of heparin into contact therewith to form ionic bonds between anion groups in heparin and cation groups in the polymer, (C) a method comprising introducing amino groups or aldehyde groups into heparin, directly immobilizing substances or functional groups capable of crosslinking with the above-mentioned functional groups onto a medical device to be a substrate and covalently binding them to immobilize heparin, and (D) a method comprising binding organic cations to anion groups in heparin to make the heparin water-insoluble but soluble to a specific organic solvent and applying the heparin solution onto the medical device.”

U.S. Pat. No. 6,200,588 also discloses that “According to the method (A), however, heparin is directly eluted into blood, so that quick elution occurs in the early stage and antithrombogenic effect is soon disappears. In addition, small holes remain on the surface of the medical device after elution of heparin, thereby possibly causing formation of thrombus on the holes.”

U.S. Pat. No. 6,200,588 also discloses that “The method (B) can provide an antithrombogenic material capable of maintaining higher anticoagulant activity for a long time due to ionic bond. However, this method requires two separate steps of coating a medical device with a quaternary ammonium salt-containing polymer to be a substrate and of binding heparin to the surface of the coated medical device. This in turn increases production cost of a medical device to be in contact with blood, which should be disposable.”

U.S. Pat. No. 6,200,588 also discloses that “The method (C) aims at antithrombogenicity retained for an extended period of time by semi-permanently immobilizing heparin on the surface of a medical device. However, the heparin immobilized on the surface by a covalent bond has limited mobility and cannot bind sufficiently with antithrombin HI required for an expression of antithrombogenicity, to the point that the surface cannot exert sufficient antithrombogenicity.”

U.S. Pat. No. 6,200,588 also discloses that “The method (D) comprises dissolving water-insoluble toridecylmethylammonium chloride in isopropyl alcohol, applying the solution onto the surface of a medical device, and then bringing the surface into contact with an aqueous solution of heparin to form an ionic complex of toridecylmethylammonium and heparin on the surface, whereby to provide antithrombogenicity. Like the method of (B), this method again requires two separate steps of coating a medical device with toridecylmethylammonium chloride and of binding heparin, which is undesirable from the aspects of cost and work efficiency.”

U.S. Pat. No. 6,200,588 also discloses that “For this shortcoming to be obliterated, a method has been proposed, which comprises dissolving an ionic complex of a benzalkonium salt and heparin in isopropyl alcohol and applying the solution onto the surface of a medical device. According to this method, the ionic complex is formed first, so that a single step of coating is sufficient. In addition, this solution is sold on the market and easily available. However, benzalkonium salts are produced from an aromatic halide as a staring material, which leaves an issue with the safety of residual starting material. Furthermore, the high cytotoxicity of the resultant benzalkonium salt, as evidenced by the use thereof as a bacteriocide during operation, poses the risk of hemolysis once it elutes out into the blood. Another problem of this method is in connection with the retention of antithiombogenicity during a long-term use of the medical device, because this ionic complex has poor durability in blood.”

U.S. Pat. No. 6,200,588 also discloses that “As can be appreciated from the foregoing, known methods have, without exception, problems in at least one aspect from long-term durability of antithrombogenic effect, production efficiency, production cost and safety.”

A New Stent Design Involving a Discrete Inductor

In this section of the specification, applicants will describe certain new stents that are comprised of discrete inductors with novel configurations. These inductors may be coated with one or more of the blood-compatible materials described elsewhere in this specification.

The novel stent designs described in this section of the specification are especially suited for use with Magnetic Resonance Imaging. As is known to those skilled in the art, Magnetic Resonance Imaging (MRI) is extensively used to non-invasively diagnose patient medical problems. The patient is positioned in the aperture of a large annular magnet that produces a strong and static magnetic field. The spins of the atomic nuclei of the patient's tissue molecules are aligned by the strong static magnetic field. Radio frequency pulses are then applied in a plane perpendicular to the static magnetic field lines so as to cause some of the hydrogen nuclei to change alignment. The frequency of the radio wave pulses used is governed by the Larmor Equation. Magnetic field gradients are then applied in the 3 dimensional planes to allow encoding of the position of the atoms. At the end of the radio frequency pulse the nuclei return to their original configuration and, as they do so, they release radio frequency energy, which can be picked up by coils wrapped around the patient. These signals are recorded and the resulting data are processed by a computer to generate an image of the tissue. Thus, the examined tissue can be seen with its quite detailed anatomical features. In clinical practice, MRI is used to distinguish pathologic tissue such as a brain tumor from normal tissue.

The MRI technique most frequently relies on the relaxation properties of magnetically-excited hydrogen nuclei. The sample is briefly exposed to a burst of radiofrequency energy, which in the presence of a magnetic field puts the nuclei in an elevated energy state. As the molecules undergo their normal, microscopic tumbling, they shed this energy to their surroundings, in a process referred to as “relaxation.” Molecules free to tumble more rapidly relax more rapidly.

Differences in relaxation rates are the basis of MRI images—for example, the water molecules in blood are free to tumble more rapidly, and hence, relax at a different rate than water molecules in other tissues. Different scan sequences allow different tissue types and pathologies to be highlighted.

MRI allows manipulation of spins in many different ways, each yielding a specific type of image contrast and information. With the same machine a variety of scans can be made and a typical MRI examination consists of several such scans. One of the advantages of a MRI scan is that, according to current medical knowledge, it is harmless to the patient. It only utilizes strong magnetic fields and non-ionizing radiation in the radio frequency range. Compare this to CT scans and traditional X-rays which involve doses of ionizing radiation. It must be noted, however, that the presence of a ferromagnetic foreign body (for example, shell fragments) in the patient, or a metallic implant (like surgical prostheses, or pacemakers) can present a (relative or absolute) contraindication towards MRI scanning: interaction of the magnetic and radiofrequency fields with such an object can lead to mechanical or thermal injury, or failure of an implanted device.

Even if implanted medical devices pose no danger to the patient, they may prevent a useful MR image from being obtained, due to their perturbation of the static, gradient and/or radio frequency pulsed magnetic fields and/or the response signal from the imaged tissue. Examples of problems encountered when attempting to use MRI to image tissue adjacent to implanted medical devices are discussed in U.S. Pat. No. 6,712,844, the entire disclosure of which is hereby incorporated by reference into this specification. U.S. Pat. No. 6,712,844 states “While researching heart problems, it was found that all the currently used metal stents distorted the magnetic resonance images. As a result, it was impossible to study the blood flow in the stents which were placed inside blood vessels and the area directly around the stents for determining tissue response to different stents in the heart region.” U.S. Pat. No. 6,712,844 goes on to state “It was found that metal of the stents distorted the magnetic resonance images of blood vessels. The quality of the medical diagnosis depends on the quality of the MRI images. A proper shift of the spins of protons in different tissues produces high quality of MRI images. The spin of the protons is influenced by radio frequency (RF) pulses, which are blocked by eddy currents circulating at the surface of the wall of the stent. The RF pulses are not capable of penetrating the conventional metal stents. Similarly, if the eddy currents reduce the amplitudes of the radio frequency pulses, the RF pulses will lose their ability to influence the spins of the protons. The signal-to-noise ratio becomes too low to produce any quality images inside the stent. The high level of noise to signal is proportional to the eddy current magnitude, which depends on the amount and conductivity of the stent in which the eddy currents are induced and the magnitude of the pulsed field.”

The currents induced in implanted metallic stents, and other devices, by the incident radio frequency radiation in the MRI field create, according to Lenz's law, magnetic fields that oppose the change of the magnetic fields of the incident radiation, thereby distorting and/or reducing the contrast of the resulting image. Examples of attempts to improve the imageability of stents in MRI by incorporating resonance circuits with the stents are found, i.e., in U.S. Pat. No. 6,280,385 (“Stent and MR Imaging Process for the Imaging and the Determination of the Position of a Stent”) and U.S. Pat. No. 6,767,360 (“Vascular Stent with Composite Structure for Magnetic Resonance Imaging Capabilities”). The entire disclosure of each of these United States patents is hereby incorporated by reference into this specification.

As used in this specification, passive resonance circuit means a resonance circuit comprised of only passive circuit elements. A passive circuit element is a circuit element that contributes no energy to the circuit such as, e.g., a resistor, a conductor, a capacitor, etc. These passive circuit elements are to be distinguished from such active circuit elements as, e.g., batteries, sources of alternating current, etc.

As used in this specification, a resistor means a device that offers opposition in the form of resistance to the flow of electric current. Electronic Dictionary (1st edition), Cooke and Marcus, McGraw-Hill Book Company, Inc. (1945) p. 322.

As used in this specification, capacitor means “an electrical device consisting essentially of two conducting surfaces separated by an insulating material or dielectric. . . a capacitor stores electrical energy, blocks the flow of direct current, and permits the flow of alternating current to a degree dependent upon the capacitance and the frequency.” Electronic Dictionary (1st edition), Cooke and Marcus, McGraw-Hill Book Company, Inc. (1945) pp. 46-47.

As well known to those skilled in the art, these circuit components or elements may be discrete elements, e.g. resistor, capacitor, inductor, and the like. Alternatively, a single circuit component or element may function as one or more circuit elements. For example, a single loop coil of copper wire is passive electric circuit containing an inductor, capacitor and resistor formed from a single element.

As used in this specification, the operating frequency of a magnetic resonance imaging system means the frequency at which the magnetic resonance imaging scanner's B1 magnetic field rotates. Said frequency essentially corresponds to the precessional frequency of the proton in a hydrogen atom when in the presence of the B0 static magnetic field of the magnetic resonance imagining scanner. This frequency may be calculated by the equation f=γB0 where γ is the gyromagnetic ratio and for hydrogen protons is essentially equal to 42.57 megahertz/Tesla. In some embodiments, this may be 32 megahertz, 63.86 megahertz, 127.71 megahertz, 256 megahertz or the like.

U.S. Pat. No. 6,280,385 states in column 3, lines 2944: “These and other objects are achieved by the present invention, which comprises a stent which is to be introduced into the examination object. The stent is provided with an integrated resonance circuit that induces a changed response signal in a locally defined area in or around the stent that is imaged by spatial resolution. The resonance frequency is essentially equal to the resonance frequency of the operating frequency of the magnetic resonance imaging system. Since that area is immediately adjacent to the stent (either inside or outside thereof), the position of the stent is clearly recognizable in the correspondingly enhanced area in the magnetic resonance image. Because a changed signal response of the examined object is induced by itself, only those artifacts can appear that are produced by the material of the stent itself.” Claim 1 in column 12 of U.S. Pat. No. 6,280,385 claims: “1. A magnetic resonance imaging process for the imaging and determination of the position of a stent introduced into an examination object, the process comprising the steps of: placing the examination object in a magnetic field, the examination object having a stent with at least one passive resonance circuit disposed therein; applying high-frequency radiation of a specific resonance frequency to the examination object such that transitions between spin energy levels of atomic nuclei of the examination object are excited; and detecting magnetic resonance signals thus produced as signal responses by a receiving coil and imaging the detected signal responses; wherein, in a locally defined area proximate the stent, a changed signal response is produced by the at least one passive resonance circuit of the stent, the passive resonance circuit comprising an inductor and a capacitor forming a closed-loop coil arrangement such that the resonance frequency of the passive resonance circuit is essentially equal to the resonance frequency of the applied high-frequency radiation and such that the area is imaged using the changed signal response.”

U.S. Pat. No. 6,767,360 states in column 2, lines 29-39: “Imaging procedures using MRI without need for contrast dye are emerging in the practice. But a current considerable factor weighing against the use of magnetic resonance imaging techniques to visualize implanted stents composed of ferromagnetic or electrically conductive materials is the inhibiting effect of such materials. These materials cause sufficient distortion of the magnetic resonance field to preclude imaging the interior of the stent. This effect is attributable to their Faradaic physical properties in relation to the electromagnetic energy applied during the MRI process.” U.S. Pat. No. 6,767,360 further states in column 2, lines 50-64: “In German application 197 46 735.0, which was filed as international patent application PCT/DE98/03045, published Apr. 22, 1999 as WO 99/19738, Melzer et al (Melzer, or the 99/19738 publication) disclose an MRI process for representing and determining the position of a stent, in which the stent has at least one passive oscillating circuit with an inductor and a capacitor. According to Melzer, the resonance frequency of this circuit substantially corresponds to the resonance frequency of the injected high-frequency radiation from the magnetic resonance system, so that in a locally limited area situated inside or around the stent, a modified signal answer is generated which is represented with spatial resolution. However, the Melzer solution lacks a suitable integration of an LC circuit within the stent.” Claims 1 and 2 in column 9 of U.S. Pat. No. 6,767,360 claim: “1. A stent adapted to be implanted in a duct of a human body to maintain an open lumen at the implant site, and to allow viewing body properties outside and within the implanted stent by magnetic resonance imaging (MRI) energy applied external to the body, said stent comprising a metal scaffold, and an electrical circuit resonant at the resonance frequency of said MRI energy integral with said scaffold. 2. A stent adapted to be implanted in a duct of a human body to maintain an open lumen at the implant site, said stent comprising a tubular scaffold of low ferromagnetic metal, and an inductance-cpacitance (LC) circuit integral with said scaffold, said LC circuit being geometrically structured in combination with said scaffold to be resonant at the resonance frequency of magnetic resonance imaging (MRI) energy to be applied to said body to enable MRI viewing of body tissue and fluid within the lumen of the stent when implanted and subjected to said MRI energy.”

Both U.S. Pat. Nos. 6,280,385 and 6,767,360 teach the incorporation of LC resonant circuits with stents to improve the imageability of such stents in MRI. However, in addition to a resonant frequency, resonant circuits are characterized by a Q factor which is a measure of the bandwidth of the current peak amplitude at the resonant frequency and depends upon the total resistance R of the resonant circuit. If the Q factor is too high, indicating a highly tuned, narrow bandwidth and high peak current at resonance, the induced current in the circuit and resultant enhanced electromagnetic signal will cause the MR image to be too bright with accompanying loss of detail. Applicants have discovered that imageability of stents may be optimized by incorporating RLC circuits with an optimized Q factor. In addition to the inductance L and capacitance C, resistance R must be selected for optimum imageability.

Applicants have also discovered that the peak or central resonance of the circuit need not be the same nor essentially the same as the resonance frequency of the hydrogen's proton in the MRI scanner's static magnetic field, e.g. 63.86 MHz in a 1.5 Tesla static field. The resonance peak or central resonance frequency of the system (stent with resonance circuit) may be higher or lower than the resonance frequency of the hydrogen's proton provided that the bandwidth is sufficiently broad to include the resonance frequency of the hydrogen's proton.

Applicants have also discovered a unique method of implementing the circuit onto and around the stent's structural frame by utilizing a combination of both electrically insulated and flexible wires and thin films having various conductivity and dielectric properties which eliminates breakage of the circuit due to the flexing of the stent during the deployment of the stent in a patient.

In one embodiment of applicants' invention, to be described later in this specification, a plurality of coated layers is disposed on an implanted device and electrically connected to flexible wires. The material and electrical parameters of the coated layers and wires are chosen and the geometry of the coated layers is arranged so that incident electromagnetic radiation induces currents in the coated layers and wires that enhances magnetic resonance imaging of the device included substances.

FIG. 2 is a schematic diagram of a medical stent 301 augmented by a circuit. As is known to those skilled in the art, a stent is an expandable wire mesh tube that is inserted into a lumen structure of the body to keep it open. Stents are used in diverse structures in the body such as the esophagus, trachea, blood vessels, and the like. Prior to use, a stent is collapsed to a small diameter. When brought into place, it is expanded either by using an inflatable balloon or is self-expending due to the elasticity of the material. Once expanded, the stent is held in place by its own tension.

Stents are usually inserted by endoscopy or other procedures less invasive than a surgical operation. Stents are typically metallic, for example, stainless steel, alloys of nickel and titanium, or the like and are therefore electrically conducting.

Referring again to FIG. 2, and to the preferred embodiment depicted therein, it will be seen that the stent assembly 300 comprises a stent structure 301 formed by a plurality of stent struts 302 which form rings 303 in a zigzag pattern. By way of demonstration, individual stent struts 302 connect to one another form a cylindrical mesh-like configuration as the stent structure 301. In some embodiments, a stent structure may be manufactured by a laser cutting process from a single cylindrical portion of material. It is to be understood that the particular zigzag pattern of the stent structure 301 in FIG. 2 is for illustrative purposes only. Other patterns for the stent structure are utilized in stent manufacturing and it is to be understood that the invention herein described applies to all patterns of stent structures.

Referring again to FIG. 2, rings 303 are connected together by bridges 304 to form a cylinder shaped stent structure 301. Such structures may be (and have been) formed by laser cutting a tube into the stent structure. Around the stent structure 301 is wrapped an electrically insulative conductive wire 310 which forms a loosely wound inductor.

In one embodiment, the electrically insulative conductive wire 310 is coated with one or more of the blood compatible materials described elsewhere in this specification. In another embodiment (not shown), the electrically insulative wire 310 is weaved in and out of the stent 301 structure's struts 302.

FIG. 2A is an expanded sectional view of section 330 of stent 300 (see FIG. 2). Referring to FIG. 2A, it will be seen that a resistor 332 is placed in series with a capacitor 340 and inductor ends 320 and 324.

Referring again to FIG. 2, and to the embodiment depicted therein, the electrically insulative wire 310 at one end 307 of the stent 301 bends around 312 to form a return portion 314 of the wire 310 which run back along the stent structure 301 to the other end 305 of the stent structure 301 from which it started. As the wire return portion 314 cross over the wire 310 at locations 316, the wire return portion 314 may cross over or under the wire loop 310. In one embodiment, the return portion of the wire 314 alternates over and under the wire 310.

A stent end 305 is fabricated during the stent fabrication process to create a staging area 306. As is well known to those skilled in the art, a staging area is a portion of a structure onto which components of an assembly may be positioned, attached, fabricated on to, and the like. In one embodiment staging area 306 is a portion of a stent strut. In another embodiment staging area 306 is a portion of a stent strut whose width is greater than other stent struts. In another embodiment staging area 306 is a portion of the area at which two struts merge. In another embodiment staging area 306 is a portion of the area where two struts merge that has a greater surface area than other areas where two struts merge. As will be apparent to those skilled in the art there will be many configurations possible.

Onto the staging area 306 are layered materials which form a capacitor 340 with electrical connection tabs 322 and 326. As used in this specification, electrical connection tabs may be any portion of an electronic component, e.g., a tab, a lead, a wire, conductive films, and the like, designed to facilitate a means to electrically connect said component to other electrical components. One end 320 of the wire 310 is electrically connected to connection tab 322. The other end 324 of the wire 310 is electrically connected to the capacitor's 340 other connection tab 326. An RLC (resistor, inductor, capacitor) is thus formed. However, it is to be recognized that the entire stent assembly 300 forms a single electrical system which can not be classified as a simple RLC circuit because of the mutual inductive coupling between the inductor 310 formed by the wire 310 and the stent structure 301 and because an additional distributive capacitance is formed between the stent structure 301 and the electrically insulative wire 310. Therefore, the terms “tuned”, “tuned circuit”, “tuning” and “tuning the circuit” refers to the adjustment of the of the resistive, inductive and capacitive properties of the entire stent assembly 300.

In one embodiment, and referring again to FIG. 2, one resistive element of the stent assembly 300 is the wire 310. In one embodiment, the resistance value of resistive element 310 is controlled by adjusting the cross sectional area of the wire (not shown). In another embodiment, the resistance value of resistive element 310 is controlled by the selection of the material type. In another embodiment, the resistive element 310 resistance values is controlled by both the cross sectional area of the wire 310 and by the selection of the material.

In one embodiment a resistor is fabricated onto the staging area 306 and is connected in series to the wire 310 and the capacitor 340; see, e.g., FIG. 2A and resistor 332. Thus the total resistance of the circuit is the sum of the resistance of the wire 310 and the resistance value of the resistor in series. In another embodiment, the total resistance of the circuit is the sum of the resistance of the wire 310 and the resistance value of the resistor in series and the resistance of the material (see element 132 and 136 in FIG. 2) used to attach the wire 310 to the capacitor and, in one embodiment, to a resistor (not shown).

FIG. 3 is a schematic illustration of a stent assembly 400 comprising a stent structure 402 and an electrically insulative wire 410 wrapped around the stent structure 402. A capacitive element 440 (see FIG. 4 and accompanying text for details) is fabricated onto a stent strut between stent strut points 430, 432 by forming layers of conductive and dielectric materials (not shown but see FIG. 4 and accompanying text for details) onto the strut 430, 432. The electrically insulative wire 410 wraps around the stent structure 402 and at one end of the stent 450 bends around 412 and to form a return wire 414 which runs along the length of the stent 402 to return to the starting end 452 of the stent 402. The two ends of the wire 416 and 418 are connected to the capacitor 440.

In another embodiment, and referring to FIG. 3, a capacitor 440 is formed over one or more stent struts.

FIG. 4 is a schematic sectional view of a stent 500 and, in particular, of one end thereof (for example, see element 452 of FIG. 3). As will be apparent, the stent assembly 500 is comprised of stent struts 510 (shown as a circle 510 for ease of simplicity of representation). Each of stent struts 510 is comprised of a metallic stent strut material onto which an insulative material 514 is preferably applied to the stent strut 510 outer surface. In one embodiment, not shown, the insulative material 514 is applied to all surfaces of the stent strut 510.

It is preferred that the insulative material 514 have a resistivity of at least 1×10¹² ohm-centimeters and, more preferably, at least about 1×10¹³ ohm centimeters. By way of illustration and not limitation, the insulative material 514 may be, e.g., aluminum nitride, parylene, natural and/or synthetic polymeric material, and the like.

Referring again to FIG. 4, and to the embodiment depicted therein, a conductive material 516 is preferably applied to a portion of the outer surface of the insulative material 514 only on the stent's end 510. In one embodiment, conductive material 516 extends over several stent struts.

It is preferred that the conductive material 516 have a resistivity of less than about 1×10⁻⁹ ohm-meters and, more preferably, 3×10⁻⁸ ohm-meters and, even more preferably, less than about 2.8×10⁻⁸ ohm-meters. In one embodiment, the conductive material has a resistivity of less than about 2×10⁻⁸ ohm-meters. In another embodiment, the conductive material has a resistivity of less than about 1.8×10⁻⁸ ohm-meters In another embodiment, the conductive material has a resistivity of less than about 1.8×10⁻⁷ ohm-meters

Referring again to FIG. 4, the conductive material 516 may be, for example, copper or silver or gold or the like.

In the embodiment depicted in FIG. 4, a dielectric material 518 is preferably applied over a portion of the conductive material 516. In one embodiment, it is preferred that dielectric material 518 have a relative dielectric constant of from about 2 and 300 and, more preferably, from about 2 to 4. Some suitable dielectric materials include, e.g., aluminum nitride, barium titanate, and the like.

Referring again to FIG. 4, a second conductive layer 520 is applied over the dielectric material 518; the conductive material used layer 520 may be identical to, similar to, or different from the conductive material 516. Conductive wire ends 530 and 534 are preferably electrically attached to the conductive layers 516 and 520, by conductive connection materials 532 and 536, respectively. Materials 532 and 536 may be, for example, solder or a conductive epoxy and the like.

Referring again to FIG. 4, and to the preferred embodiment depicted therein, a biocompatible material 540 is applied to the outer surface of the stent structure 500. In one embodiment, the biocompatible material 540 forms a hermetically sealed coating on the outer surface of the stent structure 500 that protects the stent structure from the entry of outside agents, such as, e.g., gas, blood, etc. One may produce such hermetically sealed coatings by means well known to those skilled in the art. Reference may be had, e.g., to U.S. Pat. No. 4,518,628 (hermetic coating by heterogeneous nucleation thermochemical deposition), U.S. Pat. No. 4,863,576 (hermetic coating of optical fibers), U.S. Pat. No. 5,246,734 (amorphous silicon heremetic coatings), and the like. The entire disclosure of each of these U.S. Pat. Nos. is hereby incorporated by reference into this specification.

In one preferred embodiment, the biocompatible material 540 forms an impermeable coating. Means for forming such a biocompatible, impermeable coating are well known to those skilled in the art.

By way of illustration and not limitation, one may use the biocompatible, impermeable coating described in U.S. Pat. No. 6,858,220, the entire disclosure of which is hereby incorporated by reference into this specification. This patent claims (in claim 1) “1. A microfluidic delivery system for the transport of molecules comprising: a substrate; a reservoir in said substrate for containing the molecules; a fluid control device controlling release of said molecules from said reservoir; and a thin film inert impermeable coating applied to said substrate.” Claim 2 further describes “2. The microfluidic delivery system according to claim 1 wherein said thin film inert impermeable coating is biocompatible.”

At columns 1-2 of U.S. Pat. No. 6,858,220, there are described other “prior art” coatings that are both impermeable and biocompatible. In the paragraph starting at lines 19 of Column 1, it is disclosed that “Implantable microfluidic delivery systems as the delivery devices of Santini, et al. (U.S. Pat. No. 6,123,861) and Santini, et al. (U.S. Pat. No. 5,797,898) or fluid sampling devices, must be impermeable and they must be biocompatible. The devices must not only exhibit the ability to resist the aggressive environment present in the body, but must also be compatible with both the living tissue and with the other materials of construction for the device itself. The materials are selected to avoid both galvanic and electrolytic corrosion.”

U.S. Pat. No. 6,858,220 also discloses that (in the paragraph beginning at line 29 of Column 1) “In microchip drug delivery devices, the microchips control both the rate and time of release of multiple chemical substances and they control the release of a wide variety of molecules in either a continuous or a pulsed manner. A material that is impermeable to the drugs or other molecules to be delivered and that is impermeable to the surrounding fluids is used as the substrate. Reservoirs are etched into the substrate using either chemical etching or ion beam etching techniques that are well known in the field of microfabrication. Hundreds to thousands of reservoirs can be fabricated on a single microchip using these techniques.

U.S. Pat. No. 6,858,220 also discloses that (in the paragraph beginning at line 41 of Column 1) “The physical properties of the release system control the rate of release of the molecules, e.g., whether the drug is in a gel or a polymer form. The reservoirs may contain multiple drugs or other molecules in variable dosages. The filled reservoirs can be capped with materials either that degrade or that allow the molecules to diffuse passively out of the reservoir over time. They may be capped with materials that disintegrate upon application of an electric potential. Release from an active device can be controlled by a preprogrammed microprocessor, remote control, or by biosensor. Valves and pumps may also be used to control the release of the molecules.”

U.S. Pat. No. 6,858,220 also discloses that (in the paragraph beginning at line 53 of Column 1) “A reservoir cap can enable passive timed release of molecules without requiring a power source, if the reservoir cap is made of materials that degrade or dissolve at a known rate or have a known permeability. The degradation, dissolution or diffusion characteristics of the cap material determine the time when release begins and perhaps the release rate.:

U.S. Pat. No. 6,858,220 also discloses that (in the paragraph beginning at line 60 of Column 1) “Alternatively, the reservoir cap may enable active timed release of molecules, requiring a power source. In this case, the reservoir cap consists of a thin film of conductive material that is deposited over the reservoir, patterned to a desired geometry, and serves as an anode. Cathodes are also fabricated on the device with their size and placement determined by the device's application and method of electrical potential control. Known conductive materials that are capable of use in active timed-release devices that dissolve into solution or form soluble compounds or ions upon the application of an electric potential, including metals, such as copper, gold, silver, and zinc and some polymers.”

U.S. Pat. No. 6,858,220 also discloses that (in the paragraph beginning at line 5 of Column 1) “When an electric potential is applied between an anode and cathode, the conductive material of the anode covering the reservoir oxidizes to form soluble compounds or ions that dissolve into solution, exposing the molecules to be delivered to the surrounding fluids. Alternatively, the application of an electric potential can be used to create changes in local pH near the anode reservoir cap to allow normally insoluble ions or oxidation products to become soluble. This allows the reservoir cap to dissolve and to expose the molecules to be released to the surrounding fluids. In either case, the molecules to be delivered are released into the surrounding fluids by diffusion out of or by degradation or dissolution of the release system. The frequency of release is controlled by incorporation of a miniaturized power source and microprocessor onto the microchip.”

U.S. Pat. No. 6,858,220 also discloses that (in the paragraph beginning at line 21 of Column 2) “One solution to achieving biocompatibility, impermeability, and galvanic and electrolytic compatibility for an implanted device is to encase the device in a protective environment. It is well known to encase implantable devices with glass or with a case of ceramic or metal. Schulman, et al. (U.S. Pat. No. 5,750,926) is one example of this technique. It is also known to use alumina as a case material for an implanted device as disclosed in U.S. Pat. No. 4,991,582. Santini, et. al. (U.S. Pat. No. 6,123,861) discuss the technique of encapsulating a non-biocompatible material in a biocompatible material, such as poly(ethylene glycol) or polytetrafluoroethylene-like materials. They also disclose the use of silicon as a strong, non-degradable, easily etched substrate that is impermeable to the molecules to be delivered and to the surrounding living tissue. The use of silicon allows the well-developed fabrication techniques from the electronic microcircuit industry to be applied to these substrates. It is well known, however, that silicon is dissolved when implanted in living tissue or in saline solution.”

In one preferred embodiment, the biocompatible material 540 has a dielectric constant of from about 1.5 to about 10. In one aspect of this embodiment, the biocompatible material has a dielectric constant of from about 2 to about 4.

Referring again to FIG. 4, and to the preferred embodiment depicted therein, it will be seen that a biocompatible material 541 is preferably applied to the inner surface of the stent structure 500. Materials 540 and 541 may be the same material, or a different material. In one embodiment, either or both of the 540 and/or 541 is a drug-eluting material.

In one embodiment, and referring again to FIG. 4, the electrical wire used (see elements 530, 534) has a circular cross section geometry. In another embodiment (not shown, but see FIG. 6), the electrical wire used has essentially a rectangular cross section geometry. An increase in the width of the rectangular cross section provides an increase in the cross-sectional area without increasing the radial dimension of the resulting stent assembly 500. It is well known that increasing the cross sectional area of the wire will decrease the electrical resistivity of the wire. Thus the resistance of the circuit defined around the stent can be adjusted by the selection of the wire's cross sectional geometry.

FIGS. 5A-5D illustrate other ways that stent 500 may be configured with an electrically insulating wire can be wound about a stent (illustrated as a cylinder for clarity) to form one or more inductive coils. In the embodiments depicted in FIGS. 5A and 5B, a single wire 582 is wrapped along the stent 580 length. Wire 582 may be wrapped one or more times along the stent 580 to form multi-turn coils.

FIG. 5C illustrates the use of two different electrically insulative wires, wires 584 and 586, wrapped along the stent 580 to form two different inductive coils and to become parts of two different electrical circuits. In one embodiment, the resonance circuit of which one of the two coils is tuned to resonate at a frequency f1 while the resonance circuit of which the other coil is a component is tuned to resonate at a frequency 2×f1. In another embodiment the two circuits are tuned to two non harmonic frequencies. In another embodiment the two circuits are tuned to other harmonic frequencies of each other. In another embodiment the two circuits are tuned to the same frequency.

As used in this specification, a harmonic frequency means a positive integer multiple of a given frequency. As used in this specification, a non-harmonic frequency means a frequency that is not a positive integer multiple of the given frequency.

FIG. 5D illustrates two different wires 588 and 590 wrapped along the stent 580, thereby forming two different inductive coils that have an orientation of about 90 degrees from one another.

FIG. 6 is a schematic of an assembly 650 disposed on a stent's strut 652. In the embodiment depicted in FIG. 6, stent strut 652 may consist of only one such strut, and/or it may comprise two ore more consecutive struts; alternatively, in the case where the stent's structural design is not composed of struts, “strut 652” may be a segment of the stent's mesh.

Referring to FIG. 6, and in the preferred embodiment depicted therein, an insulative coating 654 is applied to the stent's strut 652; this insulating coating may have the properties described elsewhere with regard to 514 including, e.g., biocompatibility and/or impermeability. A conductive material 656 is formed on the outer surface of the stent strut 652 over the insulating material 654; this conductive material may be identical to and/or similar to conductive material 516; and, in the embodiment depicted, it extends only along a portion of the stent's struts 652. A dielectric material 658 is applied over a portion the conductive material 656; and it may be similar to dielectric coating 518.

Referring again to FIG. 6, second conductive material 660 (which may be similar or identical to conductive material 516) is applied over a portion of the dielectric material 658. Rectangular cross section wire ends 666 and 662 are electrically attached to the conductive materials 656 and 660, respectively be conductive material 668 and 664, respectively. This assembly 650 thus forms a capacitive element on a stent's strut 652 to which the wire of the inductor coil loops (see FIG. 2, 3 and 5) are attached.

In one embodiment, not shown, multiple capacitor assemblies 650 are manufactured on multiple stent struts 652.

FIG. 7 is a schematic of an assembly 770 manufactured around a portion of a stent's strut 772. An electrically insulative material 774 (which may be similar to or identical to insulative 514) is applied to a portion of a stent strut 772. A first conductive material (which may be similar to or identical to conductive material 516) 776 is applied over the insulative material 774 and continuously around a portion of the stent's strut 772. A dielectric material 778 (which may be identical to or similar to dielectric material 518) is applied over a portion of the first conductive material 776. A second conductive material 780 (which may be identical to or similar to conductive material 516) is applied over a portion of the dielectric material 778, thus forming a capacitor continuously around a portion of a stent's strut 772. Wire ends 786 and 782 are electrically connected to the conductive materials 776 and 780 by conductive attachment materials 788 and 784, respectively. Attachment materials 788 and 784 may be, for example, solder or conductive epoxy or the like.

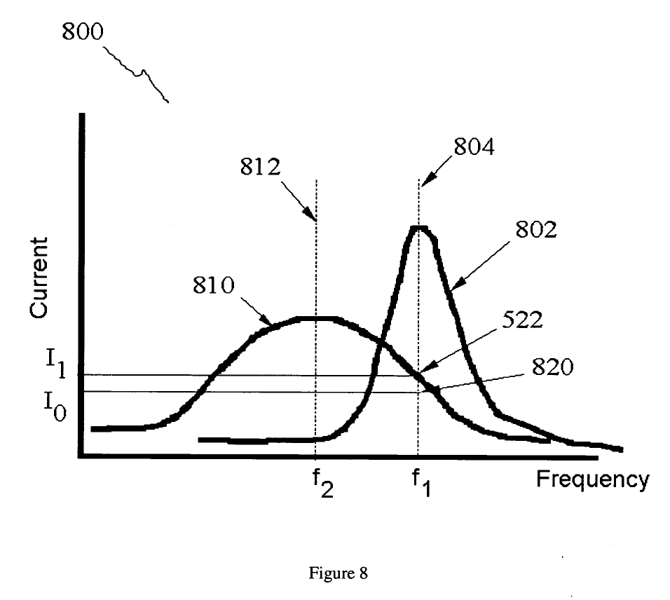

FIG. 8 is a graph 800 showing the Current versus Frequency response of two differently tuned stent assembles; curve 810 corresponds to the assembly 300 of FIG. 2, and curve 802 also corresponds to the assembly 300 of FIG. 2. The y-axis is the current induced in the wire inductive coil element (for example element 310 of FIG. 2) when the stent assembly (for example 300 in FIG. 2) is subjected to an oscillating magnetic field (for example, the rotating, pulsed magnetic field of an MRI scanner). The frequency of the oscillating magnetic field is plotted along the x-axis. The induced current plotted requires the full stent system as defined elsewhere in this specification.

Whereas the imageability of stents may be optimized by incorporating RLC circuits, the ability to select resistance values directly enhances imageability. The resistance value may be modified to achieve the desired response of the stent system and in particular, the bandwidth and the intensity of the response. Thus, an advantage is achieved by providing an additional parameter to modify, in addition to the capacitor and inductor values, the response of the system to achieve the maximum imageability and detail of the stent in the body. Another significant advantage is achieved in providing the imageability of the stent's lumen as positioned in a patient, in vivo, allowing for therapeutic monitoring of the stent in vivo over time.

Referring again to FIG. 8, traces 810 and 802 are induced current responses in the added wires (for example 310 of FIG. 1) of two differently-tuned sent assembles. In both cases, the stent structure and the inductor coil (for example, 310 in FIG. 1) of the stent assembly are the same. Trace 802, representing the induced current response for stent assemble #1, has a peak induced current resonance frequency 804, labeled “f1” in the graph. Trace 810, representing the induced current response of stent assembly #2, has a peak induced current resonance frequency 812, labeled “f2” in the graphs, and which, in this case, is lower then the resonance frequency “f1” of stent assembly #1. For the case illustrated in FIG. 8, “f1” is also the precessional frequency of the hydrogen proton in the static magnetic field B0 of the MRI scanner into which the stent assembly is placed. That is, stent assembly #1 is tuned to the resonance frequency of the MRI scanner. In the case of a 1.5 Tesla MRI scanner this frequency is 63.86 MHz (mega-hertz), approximately. A minimum induced current 820 (and labeled “I0”) is determined to be the minimum induced current in stent assembly #1 and stent assembly #2 inductive coils (for example, 310 of FIG. 2) which enhances the MRI imageability of the stent assembly's lumen.

As can be seen in the graph of FIG. 8, stent assembly #2, which has a lower resonance frequency “f2” than stent assembly #1 resonance frequency “f1”, still has a sufficiently large induced current response 822 (also labeled “I1”) at the higher frequency “f1” to enhance the imageability of the stent's lumen. That is, at frequency “f1” the induced current in stent assembly's #2 inductive coil (310 of FIG. 2) is larger than the minimum induced current “I0” required to enhance MR imaging of the stent's lumen even though stent assembly #2 was tuned to have a lower resonance peak frequency “f2”.

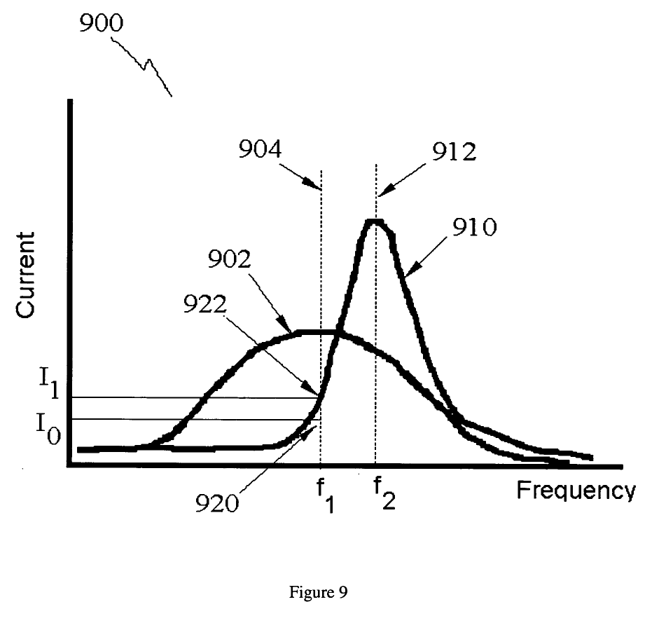

FIG. 9 is a plot of another Current versus Frequency response for two differently tuned stent assemblies, such as, e.g., differently tuned stent assemblies 300. In this case, the hydrogen resonance frequency of the MR scanner is “f1”. Stent assembly #1 response (trace 902) is tuned to have 904 (labeled “f1”) as its resonance peak current response. Stent assembly #2 response (trace 910) is tuned to have a different, higher resonance peak current response 912 (labeled “f2”). Stent assembles #1 and #2 (for example stent 300 of FIG. 2) have the same stent structures (for example 303 of FIG. 2) and the same inductive coil design (for example 310 of FIG. 2). There is a minimum induced current required 920 (labeled “I0”) above which stent lumen imageability is noticeably enhanced. As can be seen, for stent assembly #2, the induced current response 922 labeled “I1” at the frequency “f1” is larger than the minimum required induced current “I0” and is therefore sufficiently large to enhance the imageability of the stent's lumen even though the tuned resonance peak frequency “f2” of stent assembly #2 is higher than the frequency “f1”.

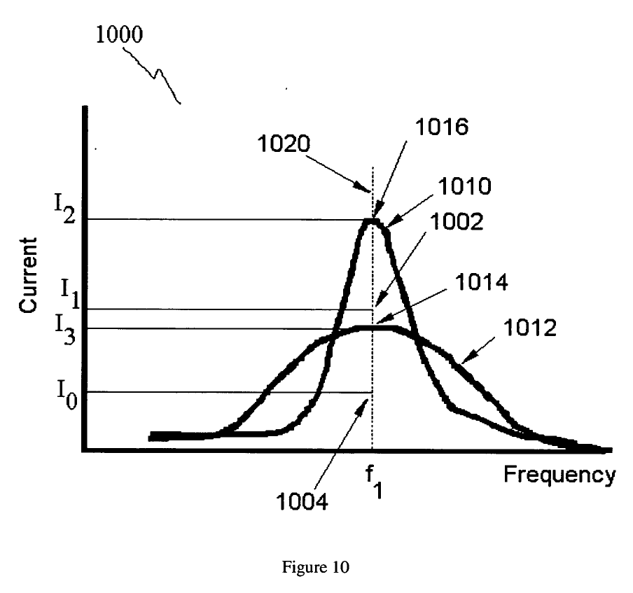

FIG. 10 shows a plot 1000 of the Current versus Frequency response of two different stent assemblies such as, e.g., stent assembly 400 of FIG. 3. In this case, the hydrogen resonance frequency of the MR scanner is “f1”. Stent assembly #1 response (trace 1010) is tuned to have 1020 (labeled “f1”) as its resonance peak current response. Stent assembly #2 response (trace 1012) is tuned to have the same resonance peak current response 1020 (labeled “f1”). Stent assembles #1 and #2 (for example 400 of FIG. 3) have the same stent structures (for example 402 of FIG. 3) and the same inductive coil design (for example 410 of FIG. 3). There is a minimum induced current required 1004 (labeled “I0”) above which stent lumen imageability is noticeably enhanced. There is also a maximal induced current 1002 (labeled “I1” ) above which the induced current is so large that the quality of the image of the stents lumen is significantly degraded. As can be seen from the plot in FIG. 10, for stent assembly #1, the induced current response “I2” at the frequency “f1” is larger than the maximum limit set for imageability of the stent's lumen. The quality of the image will therefore be degraded. However, for stent assembly #2, the induced current response “I3” at the frequency “f1” is between the minimum “I0” and maximum “I1” current range set for imageability of the stent's lumen and will therefore result in an enhanced image of the stent's lumen of acceptable quality. In this case, the resistance for the circuit of stent assembly #2 is larger than the resistance for the circuit of stent assembly #1, with all other circuit parameters being equal which lowers the induced current in the wire inductor of stent assembly #2.

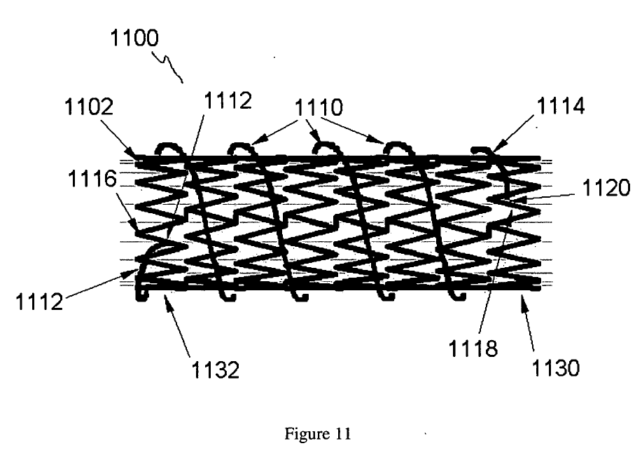

FIG. 11 is a schematic of a stent assembly 1100 comprising a stent structure 1102 and an electrically insulative wire 1110 wound around the stent structure 1102 to form an inductive coil 1110. In this embodiment, the wire begins at one end 1130 of the stent structure 1102 and is wrapped around the stent to the other end 1132 of the stent structure 1102. The wire end 1114 is electrically connected to a single terminal capacitor (not shown) on stent strut 1118 at the connection point 1120. The other end of the wire 1112 is connected to another single terminal capacitor (not shown) on stent strut 1116 at connection point 1112. In this way, the return wire (for example 314 of FIG. 2) is not required.

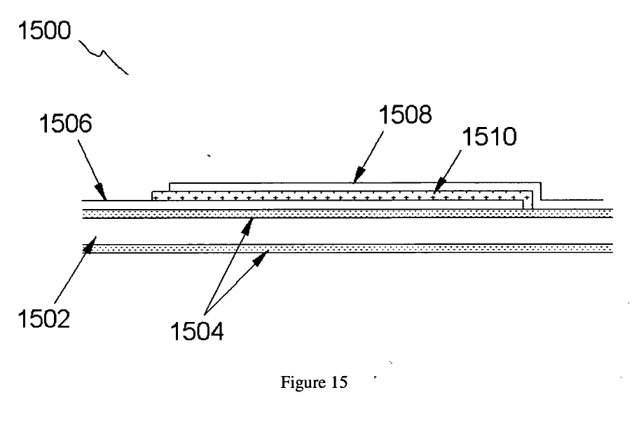

FIG. 15 depicts a portion of a stent assembly 1500 wherein a capacitor is formed without attachment to other circuit components. In this embodiment, a portion of a stent structure 1502 is layered with an electrically insulating material 1504. In one embodiment, the insulating material may be as described elsewhere in this specification. Onto a portion of the insulating material is layered a first conductive material 1506, which, in the embodiment depicted, is a portion of an inductor according to, e.g. 310 of FIG. 2. A dielectric material 1510 is layered onto a portion of conductive material 1506. A second conductive material 1508 is layered onto the dielectric material 1510. In one embodiment, the insulating material may be as described elsewhere in this specification and may be the same for the first and second conductive materials. In the embodiment depicted, second conductive material 1508 is a portion of an inductor according to, e.g. 310 of FIG. 2. Thus, a capacitor is formed in series with and inductor. Other embodiments include any one of the inductors or inductor coils disclosed in this specification.

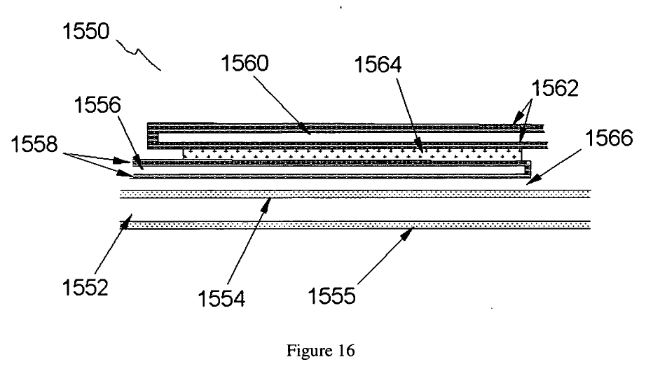

FIG. 16 depicts a portion of stent assembly 1550 wherein a capacitor is formed without attachment to other circuit components. In this embodiment, and as shown in FIG. 16, a portion of a stent structure 1552 is layered with a material 1554, 1555, which in one embodiment is an oxidation layer formed over the stent structure 1552. In another embodiment, materials 1554, 1555 are drug-eluting materials. About the vicinity of said portion of stent structure 1552 is a portion of conductive material 1556 that is covered with an electrical insulating material 1558. In one embodiment, conductive material 1556 is a portion of an inductor. A dielectric material 1564 is layered onto a portion of electrical insulating material 1558. A second conductive material 1560, surrounded by a second electrically insulative material 1562, is layered onto dielectric material 1564. A gap 1566 is formed above material 1554 and the first insulating material 1558 of first conductive material 1556. Thus, a capacitor is formed in such a way that the capacitor is not directly attached to the stent structure.

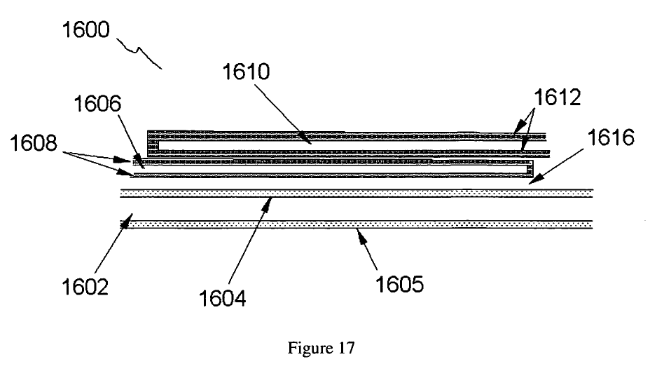

FIG. 17 depicts a portion of stent assembly 1600 wherein a capacitor is formed without attachment to other circuit components. In this embodiment, and as shown in FIG. 17, a portion of a stent structure 1602 is layered with an material 1604, 1605, which in one embodiment is an oxidation layer formed over the stent structure 1602. In another embodiment, materials 1604, 1605 are drug-eluting materials. About the vicinity of said portion of stent structure 1602 is a portion of conductive material 1606 that is covered with first electrical insulating material 1608. In the embodiment depicted, conductive material 1606 is a portion of an inductor. A second conductive material 1610, surrounded by a second electrically insulative material 1612, is layered onto first insulative material 1608. Thus, a capacitor is formed in such a way that the capacitor is not directly attached to the stent structure.

Determination of Resonant Frequency

As is known to those skilled in the art, the electrical characteristics of an electrical circuit can change depending on the environment into which the circuit is placed. For example, parasitic capacitance can form at the interface of the circuit's materials and the circuit's environment. Hence, the response, and in particular a resonance response, of a circuit or a system comprising a circuit depends on the environment into which the system is placed. Thus, a system that resonates at one frequency in an air environment may resonate at a different frequency in an essentially liquid and/or semi-liquid environment of a patient's body.

In the process of this invention, certain resonance characteristics are achieved by the stent system. As used in this specification, stent system means a stent assembly, an electrical circuit in the proximity of and/or in contact with a portion of the stent, and the tissue and fluids contained within and around the stent assembly when the stent assembly is positioned into a patient, or substitute materials for the patient's tissues and fluids that have essentially the same electrical and magnetic properties as said patient's tissues and fluids, and in some cases, a container to contain said stent assembly, electrical circuit and substitute materials within a measurement system.

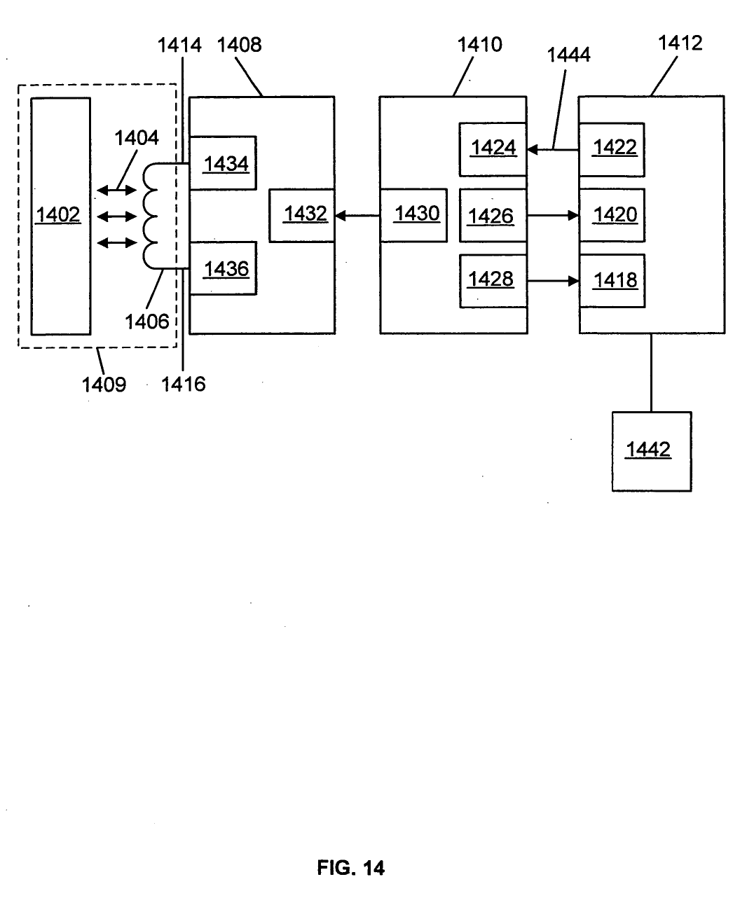

In one embodiment, the stent system comprises a vascular stent. It should be understood that the stent is not limited to a vascular stent and may be any of the stents described in the prior art for other parts of the body. In another embodiment the stent system comprises a vascular stent and an electrical circuit in the proximity of and/or in contact with a portion of the vascular stent. In another embodiment, the stent system comprises a vascular stent, an electrical circuit in the proximity of and/or in contact with a portion of the vascular stent, and the tissue and fluids contained within and around the vascular stent when the stent is positioned into a patient. In another embodiment, the stent system comprises a vascular stent, an electrical circuit in the proximity of and/or in contact with a portion of the vascular stent, and substitute materials which can be substituted for the patient's tissues and fluids and have essentially the same electrical and magnetic properties as said patient's tissues and fluids. In another embodiment, the stent system comprises a vascular stent, an electrical circuit in the proximity of and/or in contact with a portion of the vascular stent, substitute materials which can be substituted for the patient's tissues and fluids and have essentially the same electrical and magnetic properties as the said patient's tissues and fluids, and a container to contain said stent, electrical circuit and substitute materials within a measurement system, e.g., as depicted in FIG. 14. In one embodiment, the container of the stent system is comprised of a glass beaker. In another embodiment, the container of the stent system is a Pyrex container. In yet another embodiment, the container of the stent system is comprised of a polymer material, e.g., a plastic, nylon or the like. In one embodiment, said container is a nonconductive and nonmagnetic container suitable for containing liquids at essentially room temperature.

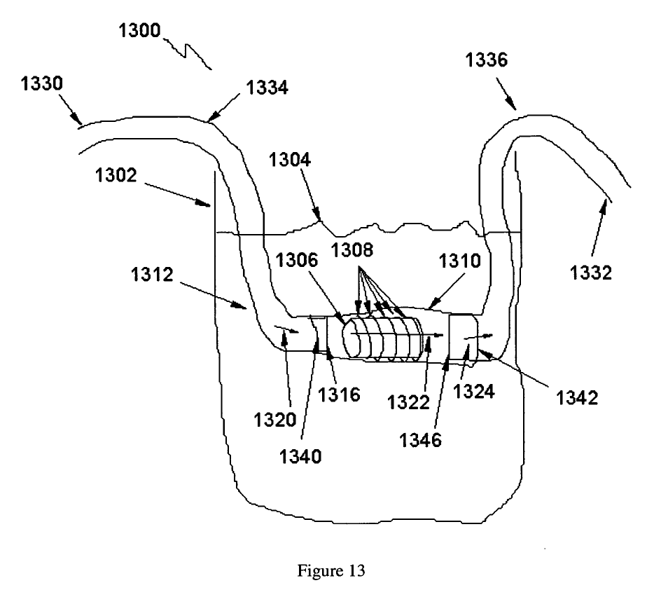

FIG. 13 depicts one embodiment of a stent system 1300 comprising a vascular stent 1306 submerged in a material 1304 contained in a container 1302. The container 1302 may be, e.g., a glass beaker, a plastic container or other non-electrically conductive and nonmagnetic container suitable for containing material 1304 in a room temperature environment. Material 1304 may be, e.g., a liquid material, a gelled material or the like. In one embodiment, material 1304 may be blood. In another embodiment material 1304 may be a material with essentially the same electrical and magnetic properties of muscle tissue.

Continuing to refer to FIG. 13 and to the embodiment depicted therein, stent 1306 is in the proximity of an RLC circuit 1308 which may be, e.g. one of the circuit configurations disclosed in this application. The stent 1306 and RLC circuit 1308 is positioned within a tubular material 1310. Material 1310 may be, e.g. a portion of an animal artery, or other vascular material, or a vascular substitute which has essentially the same electromagnetic properties of human vascular tissue. Material 1310 is attached to tubes 1334 and 1336. Material 1310 has an end 1340 attached to the end 1316 of tubing 1334. Material 1310 has an end 1342 attached to the end 1346 of tubing 1336. A pump (not shown and not part of the stent system) pumps a liquid 1320, 1322, 1342, through the tubing 1330, through the material 1310 and through the tubing 1336. Said liquid may be, e.g., blood or other liquid which has essentially the same electric and magnetic properties of blood. The moving liquid 1320 passes though the tubing 1334 and enters the material 1310 to become the moving liquid 1322 which also passes through the stent 1306. Liquid 1322 passes through the material 1310 to exit the material 1310 as moving liquid 1324 and enters the tubing 1336 at tub end 1346.

The pump (not shown and not part of the stent system) may pulse the flow of liquids 1320, 1322, 1324 to simulate essentially the pulse flow of blood in a body.

The resonance characteristics of the said stent system may be determined by the test method depicted in FIG. 14 or by other conventional means known to those skilled in the art.

FIG. 14 depicts one embodiment of an impedance test apparatus suitable for determining the resonance frequency of the stent system. An Agilent Technologies, Inc. model 4395A-010 network/spectrum/impedance analyzer 1412 comprises a display and is operationally connected to an Agilent Technologies, Inc. model 43961A test impedance kit 1410 which is operationally connected to an Agilent Technologies, Inc. model 16092A test fixture 1408. Additionally and optionally an Agilent Technologies, Inc. model 85032E calibration kit 1442 may be connected to the said network/spectrum/impedance analyzer 1412 and, as is known to those skilled in the art, may be used to calibrate said Agilent Technologies, Inc. model 4395A-010 network/spectrum/impedance analyzer 1412 before a measurement is performed.

In the embodiment depicted, said Agilent Technologies, Inc. model 4395A-010 Network/spectrum/impedance analyzer 1412 RF output port 1422 is operationally connected to said Agilent Technologies, Inc. model 43961A test impedance kit 1410 RF input port 1424 by an N-N cable 1444. Further, the R connections 1426, 1420 and A connections 1418, 1428 are appropriately connected between said devices.

Said test impedance kit 1410 is operationally connected to said test fixture 1408 at the output port 1430 of said test impedance kit 1410 and port 1432 of the test fixture 1408.

A single wire wound measurement solenoid coil 1409 which operationally is an inductor 1406 comprises leads 1414 and 1416 (which are the two ends of the wire used to construct the measurement solenoid coil 1409) surrounds the stent system 1402 under test. Said leads 1414 and 1416 are electrically connected to ports 1434, 1436 of said test fixture 1408. Thus, as is known to those skilled in the art, a single port connection is operationally made to the Network/spectrum/impedance analyzer 1412.

The stent system 1402 under test inductively couples 1404 to the measurement solenoid 1409 which operationally acts as an inductor 1406, thus, and as is known to those skilled in the art, changing the impedance characteristics of the measurement solenoid coil 1409 as a function of frequency.

As is known to those skilled in the art, the radio frequency signal produced by the Agilent Technologies, Inc. model 4395A-010 network/spectrum/impedance analyzer 1412 may be set to sweep from a frequency range of about 20 megahertz to about 100 megahertz, or about 40 megahertz to about 80 megahertz, or about 10 megahertz to about 300 megahertz, or about 100 kilohertz to about 500 megahertz.

As is known to those skilled in the art, the impedance of an electrical system is in general a complex number value and may be represented as
Z=R+iX

Where R is the resistance, X is the reactance and i is the square root of negative 1. As is known to those skilled in the art, the complex number part X of the impedance Z of the measurement solenoid 1409 around stent system 1402 is in part a function of frequency and can be graphed by the Agilent Technologies, Inc. model 4395A-010 network/spectrum/impedance analyzer 1412 as a function of the swept frequency range specified such that along the x-axis is the frequency and along the y-axis is the reactance X of the impedance measured.

As is known to those skilled in the art, the Agilent Technologies, Inc. model 4395A-010 network/spectrum/impedance analyzer 1412 directly measures impedance parameters operating in the radio frequency range of about 100 kilohertz to about 500 megahertz and with about a 3% impedance accuracy. The source level is from about −0.56 decibels per milliwatt to about +9 decibels per milliwatt at device under test and a direct current bias of about 40 volt and a maximum of about 20 milliamphere and open/short/load compensation.

As is known to those skilled in the art, when the graphed reactance X crosses the x-axis a resonance condition is indicated having a frequency at the corresponding crossing point along the x-axis value.

In another embodiment, the Agilent Technologies, Inc. model 4395A-010 network/spectrum/impedance analyzer 1412 graphs the magnitude of the impedance |Z| as a function of frequency. The frequency is again along the x-axis. The magnitude of the impedance |Z| is along the y-axis. In this embodiment the resonance frequency of the stent system 1300 is the frequency at which |Z| is a maximum in the frequency range selected. It is to be understood that in any electrical system there may occur more than one resonance.

It is expressly understood that while the above discussion sets forth some preferred embodiments for implementing the invention and determining the resonance frequency, along with preferred frequency ranges of operation and apparatus configuration, any suitable implementation design could be constructed under the teachings herein and any suitable radio frequency transmission range or ranges could be used.

### EXAMPLE 1

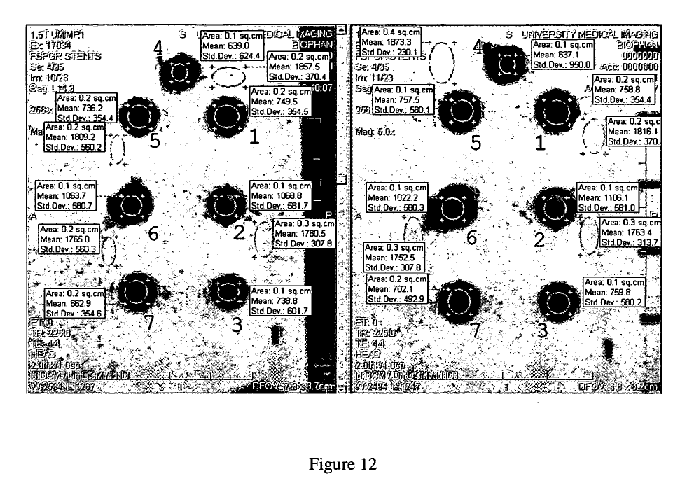

FIG. 12, and the embodiment depicted therein, shows two magnetic resonance image slices of seven stents identified as 1 through 7. The average signal intensities of selected regions are labeled in FIG. 12 as the “Mean” and appear in text boxes adjacent to the stent images in each image slice. In this example, imaging of said stents 1-7 was performed with a General Electric 1.5 Tesla MRI scanner with resonance frequency of about 63.86 megahertz using a Fast Spoiled Gradient imaging sequence. In the embodiment depicted, the stents were submerged in a vegetable oil phantom liquid. The MRI scanner's head receiver coil was used to detect the signals forming the images depicted in FIG. 12. Additional imaging parameters are listed in Table #1.

TABLE 1
Imaging parameters.

| TE | Min full | |
|---|---|---|
| TR | 225 | ms |
| Flip Angle | 90 | degrees |
| FOV | 18 | cm |
| Slice Thickness | 2 | mm |
| Spacing | 1 | mm |

| | |
|---|---|
| Freq. | 256 |
| Phase | 256 |
| Phase FOV | 1.0 |
| NEX | 1 |
| Bandwidth | 31.25 |

In this experiment, the specific absorption rates (SARs) were reported by the MRI scanner to be: Estimated SAR=0.0117, Average SAR=0.2699 and Peak SAR=0.6747.

Again referring to FIG. 12, stents 1, 3, 5, and 7 were unmodified Nitinol stents with a diameter of about 6 millimeters, a length of about 6 centimeters and a zigzag structure. Stents 2, 4, and 6 were Nitinol stents with a diameter of about 6 millimeters, a length of about 6 centimeters, and a zigzag structure, further having been augmented by electrical circuits comprising resistors, inductors, and capacitors similar to that disclosed in this patent but such that the capacitors were not affixed to the stent structure. Table #2 lists the electrical properties of these stents.

TABLE 2
Stent Assembly Electrical Properties

| | | | | | RESONANCE |
|---|---|---|---|---|---|
| | RESISTOR | INDUCTOR | CAPACITOR | | FREQUENCY |
| STENT | (Ohm) | (nanoHenries) | (picoFarads) | Q-FACTOR | (MHz) |
| 2 | 380 | 270 | 24 | 4 | 63 |
| 4 | 420 | 200 | 30 | 5.5 | 65 |
| 6 | 420 | 170 | 33 | 6 | 67.5 |

As will be apparent from the data in Table 2, none of the stents with augmented circuits were tuned to the 63.86 megahertz resonance frequency of the MRI scanner.

The stents were positioned perpendicular to the MRI scanner's static 1.5 Tesla magnetic field within the MRI scanner's head coil. The inductor coil for stent 2 was a two turn rectangular coil similar to what depicted in FIG. 5B. The inductor coil for stent 4 was an eight-turn spiral coil similar to what is shown in FIG. 2. The inductor coil for stent 6 was a four-turn spiral coil similar to what is depicted in FIG. 2.

Referring to FIG. 12, the “Mean” signal intensities inside stent 2 were 1068.8 in one imaging slice and 1106.1 in the other imaging slice. The “Mean” signal intensity inside stent 6 were 1063.7 in one imaging slice and 1022.2 in the other imaging slice. For stents 1, 3, 5 and 7 the “Mean” signal intensities from inside these stents ranged from a low of about 662.9 to a high of about 749.5 in one imaging slice and from a low of about 702.1 to a high of about 759.8 in the other imaging slice. The off resonance circuits added to stents 2 and 6 had higher signal intensity from the lumen of these stents than for stents 1, 3, 5, and 7 with no circuits.

The foregoing description details the embodiments most preferred by the inventors. Variations to the foregoing embodiments will be readily apparent to those skilled in the art and therefore, the scope of the invention should be measured by the appended claims.

1. An implantable stent assembly comprised of a stent a first insulating material, and a passive resonance circuit having an inductor and a capacitor, wherein:
(a) said first insulating material is disposed on said inductor, wherein said first insulating material is biocompatible and forms a liquid impermeable barrier around said inductor; and
(b) the resonance frequency of said stent corresponds substantially to a resonance frequency of a rotational frequency of the B1 field of the MR scanner applied by the magnetic resonance imaging system.

2. An implantable stent assembly comprised of a stent a first insulating material, and a passive resonance circuit having an inductor, a capacitor, and a resistor, wherein:
(a) said first insulating material is disposed on said inductor, wherein said first insulating material is biocompatible and forms a liquid impermeable barrier around said inductor; and
(b) the resonance frequency of said stent corresponds substantially to a resonance frequency of a rotational frequency of the B1 field of the MR scanner applied by the magnetic resonance imaging system.

3. An implantable stent assembly comprised of a stent a first insulating material, and a passive resonance circuit having an inductor, a capacitor, and a resistor, wherein:
(a) said first insulating material is disposed on said inductor, wherein said first insulating material is biocompatible, has a relative dielectric constant of from about 1.5 to about 10, and forms a liquid impermeable barrier around said inductor; and
(b) the resonance frequency of said stent corresponds substantially to a resonance frequency of a rotational frequency of the B1 field of the MR scanner applied by the magnetic resonance imaging system.

4. The stent assembly as recited in claim 3, wherein said first insulating material has a relative dielectric constant of from about 2 to about 4.

5. An implantable stent assembly comprised of an implantable conductive stent, at least one passive circuit having an inductor, a capacitor and a resistor and a first insulating material interposed between at least a portion of said implantable conductive stent and a portion of said passive circuit.

6. The implantable stent assembly recited in claim 5, wherein said first insulating material has a resistivity of at least about 1×10¹² ohm-centimeters.

7. The implantable stent assembly recited in claim 5, wherein said first insulating material comprises aluminum nitride.

8. The implantable stent assembly recited in claim 5, wherein said first insulating material comprises parylene.

9. The implantable stent assembly recited in claim 5, wherein said first insulating material is a polymeric material.

10. An implantable stent assembly comprised of an implantable conductive stent with a longitudinal axis, at least one passive circuit having an inductor, a capacitor and a resistor, an electrically insulated wire with a first end and a second end, and a first insulating material interposed between at least a portion of said implantable conductive stent and a portion of said passive circuit.

11. (canceled)

12. The implantable stent assembly as recited in claim 10, wherein said first insulating material has a relative dielectric constant of from about 2 to about 4.

13. The implantable stent assembly as recited in claim 10, wherein said first insulating material has a resistivity of at least about 1×10¹² ohm-centimeters.

14. The implantable stent assembly as recited in claim 10, wherein said first insulating material comprises parylene.

15. The implantable stent assembly as recited in claim 10, wherein said first insulating material comprises aluminum nitride.

16. The implantable stent assembly as recited in claim 10, wherein said first insulating material comprises a polymeric material.

17. (canceled)

18. (canceled)

19. (canceled)

20. (canceled)

21. (canceled)

22. The implantable stent assembly as recited in claim 75, wherein said first conductive material has a resistivity of less than about 1.8×10⁻⁷ ohm-meters.

23. The implantable stent assembly as recited in claim 75, wherein said first conductive material is selected from the group consisting of copper, silver and gold.

24. The implantable stent assembly as recited in claim 75, wherein said dielectric material has a relative dielectric constant from about 1 to about 300.

25. The implantable stent assembly as recited in claim 75, wherein said dielectric material has a relative dielectric constant from about 1.5 to about 10.

26. The implantable stent assembly as recited in claim 75, wherein said dielectric material has a relative dielectric constant from about 2 to about 4.

27. The implantable stent assembly as recited in claim 75, wherein said dielectric material comprises aluminum nitride.

28. (canceled)

29. (canceled)

30. (canceled)

31. (canceled)

32. (canceled)

33. The implantable stent assembly as recited in claim 75, wherein said second conductive material has a resistivity of less than about 1.8×10⁻⁷ ohm-meters.

34. The implantable stent assembly as recited in claim 75, wherein said second conductive material is selected from the group consisting of copper, silver and gold.

35. An implantable stent assembly comprised of an implantable conductive stent, a first insulating material, and at least one passive circuit having an inductor, a capacitor and a resistor, wherein said implantable stent assembly has at least one resonance frequency when disposed in a human body and wherein said resistor assists in producing a bandwidth for said implantable stent assembly greater than 1.0 kilohertz.

36. (canceled)

37. An implantable stent assembly comprised of an implantable conductive stent, a first insulating material, and at least one passive circuit having an inductor, a capacitor and a resistor, wherein said implantable stent assembly has at least one resonance frequency when disposed within a human body,
and wherein said resonance frequency of said implantable stent assembly disposed in a human body is within one kilohertz of the operating frequency of a magnetic resonance imaging system.

38. The implantable stent assembly as recited in claim 37, wherein said operating frequency is selected from the group of 42.57 megahertz, 63.85 megahertz, and 127.7 megahertz.

39. (canceled)

40. An implantable stent assembly comprised of an implantable conductive stent, a first insulating material, and at least one passive circuit having an inductor, a capacitor and a resistor, wherein said implantable stent assembly has at least one resonance frequency when disposed in a human body, and wherein said resonance frequency of said implantable stent assembly is a frequency that is more than one kilohertz above or below the operating frequency of a magnetic resonance imaging system.

41. (canceled)

42. (canceled)

43. The implantable stent assembly as recited in claim 10, wherein said implantable conductive stent further comprises a return wire.

44. (canceled)

45. The implantable stent assembly as recited in claim 10, wherein said electrically insulated wire comprises a coiled wire.

46. The implantable stent assembly as recited in claim 45, wherein said electrically insulated wire comprises a coiled wire with at least one loop in the shape of a spiral disposed coaxially with said implantable conductive stent.

47. The implantable stent assembly as recited in claim 10, wherein said electrically insulated wire comprises a wire with an essentially circular cross-section.

48. The implantable stent assembly as recited in claim 10, wherein said electrically insulated wire comprises a wire with a substantially rectangular cross-section.

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. The implantable stent assembly as recited in claim 10, wherein said electrically insulated wire acts as an inductor.

62. The implantable stent assembly as recited in claim 10, wherein said electrically insulated wire acts as a capacitor.

63. The implantable stent assembly as recited in claim 10, wherein said electrically insulated wire acts as a resistor.

64. The implantable stent assembly as recited in claim 10, wherein said electrically insulated wire acts as an inductor, a resistor and a capacitor.

65. The implantable stent assembly as recited in claim 10, wherein said electrically insulated wire acts as an inductor and a resistor.

66. The implantable stent assembly as recited in claim 10, wherein said electrically insulated wire acts as an inductor and a capacitor.

67. The implantable stent assembly as recited in claim 10, wherein said electrically insulated wire comprises coiled wire in a substantially rectilinear shape disposed along the longitudinal axis of said implantable conductive stent.

68. The implantable stent assembly as recited in claim 63, wherein said resistor has a resistance determined by the geometry of the cross section of a portion of said electrically insulated wire.

69. The implantable stent assembly as recited in claim 63, wherein said resistor has a resistance determined by the material of a portion of said electrically insulated wire.

70. The implantable stent assembly as recited in claim 63, wherein said resistor has a resistance determined by the geometry of the cross section and the cross sectional area of a portion of said electrically insulated wire.

71. The implantable stent assembly as recited in claim 63, wherein said resistor has a resistance determined by the cross sectional area of said electrically insulated wire.

72. The implantable stent assembly as recited in claim 62, wherein said capacitor comprises overlapping said first end and said second end of said electrically insulated wire.

73. The implantable stent assembly as recited in claim 10, wherein said first insulating material is a biocompatible material.

74. The implantable stent assembly as recited in claim 10, wherein
(a) said capacitor comprises an insulating material, a first and second conductive materials, and a dielectric material;
(b) said insulating material is disposed on at least a portion of said electrically conductive stent;
(c) said first conductive material is comprised of said first end of said electrically insulated wire;
(d) said dielectric material comprises said insulating material; and
(e) said second conductive material is comprised of said second end of said electrically insulated wire.

75. The implantable stent assembly as recited in claim 10, wherein
(a) said capacitor comprises an insulating material, a first and second conductive materials, and a dielectric material;
(b) said insulating material is disposed on at least a portion of said electrically conductive stent;
(c) said first conductive material is disposed on at least a portion of said first insulating material;
(d) said dielectric material is disposed on at least a portion of said first conductive material; and
(e) said second conductive material is disposed on at least a portion of said dielectric material.

76. The implantable stent assembly as recited in claim 10, wherein:
(a) said implantable stent assembly comprises a stent structure having at least one stent strut;
(b) said capacitor comprises a first insulating material, a first conductive material, a dielectric material and a second conductive material;
(c) said first insulating material is disposed on at least a portion of at least one stent strut and continuously around said stent strut;
(d) said first conductive material is disposed on at least a portion of said first insulating material;
(e) said dielectric material is disposed on at least a portion of said first conductive material; and
(f) said second conductive material is disposed on at least a portion of said dielectric material.

77. The implantable stent assembly as recited in claim 75, wherein said dielectric material comprises barium titanate.

78. An implantable stent assembly comprised of an implantable conductive stent with at least one passive circuit having an inductor, at least one single terminal capacitor and a resistor, wherein said stent structure comprises an electrically insulated wire, wherein said electrically insulated wire comprises a first end and a second end, wherein said first end of said electrically insulated wire is electrically connected to a single terminal capacitor on said stent structure and wherein said second end of said electrically insulated wire is electrically connected to a single terminal capacitor on said stent structure.

79. The implantable stent assembly as recited in claim 78, wherein said insulated wire is wound around said stent structure and extends from said first end of said stent structure to said second end of said stent structure to form an inductive coil.

80. The implantable stent assembly as recited in claim 78, wherein said insulated wire is woven in and around said struts of said stent structure and extends from said first end of said stent structure to said second end of said stent structure to form an inductive coil.

81. The implantable stent assembly as recited in claim 78, wherein said insulated wire wraps around said struts of said stent structure and extends from said first end of said stent structure to said second end of said stent structure.

82. The implantable stent assembly as recited in claim 10 wherein:
(a) said implantable stent structure comprises a first surface where at least one electronic component is disposed, a first end, a second end, an outer surface, at least one connector point, at least one capacitor, at least one return wire and at least one stent strut; and
(b) said capacitor comprises a portion of said outer surface of said stent structure wherein an insulating coating is disposed on a portion of said outer surface of said stent structure; a first conductive material is applied over said insulating material on said portion of said outer surface of said stent structure; a dielectric material is applied over a portion of the first conductive material on said portion of said outer surface of said stent structure; and a second conductive material is applied over a portion of the dielectric material on said portion of said outer surface of said stent structure.

83. An implantable stent assembly comprised of an implantable stent, an electrically insulated wire, a first insulating material, and at least one passive circuit having an inductor, a capacitor and a resistor, wherein said implantable stent assembly has at least one resonance frequency when disposed in a human body, and wherein
(a) said implantable stent structure comprises a first strut, a second strut, a first end, a second end, an outer surface, at least one connector point, at least two connector tabs, and at least one capacitor;
(b) said electrically insulated wire is disposed around said outer surface of said stent structure;
(c) said electrically insulated wire is disposed from said first end of said stent structure to said second end of said stent structure and bends around to return to its starting point at said first end of said stent structure, thereby forming a return wire;
(d) said first end and second end of said electrically insulated wire are electrically connected to said capacitor;
(e) said capacitor is disposed on stent structure between said first stent strut and said second stent strut;
(f) said capacitor further comprises a first conductive material, a second conductive material, a dielectric material and an insulating material; and
(g) said layer of insulating material is disposed on a portion of said outer surface of said stent structure between said first stent and said second stent; a first conductive material disposed over at least a portion of said insulating material; a dielectric material disposed over at least a portion of said first conductive material; and a second conductive material disposed over at least a portion of said dielectric material.

84. The implantable stent assembly as recited in claim 83, wherein said first conductive material is substantially the same material as said second conductive material.

85. The implantable stent assembly as recited in claim 83, wherein said first conductive material is different from said second conductive material.

86. The implantable stent assembly as recited in claim 83, wherein said electrical connection of said insulated wire and said capacitor comprises solder.

87. The implantable stent assembly as recited in claim 83, wherein said electrical connection of said insulated wire and said capacitor comprises conductive epoxy.

88. An implantable stent assembly that is disposed in a human body and comprised of an implantable conductive stent structure with an inner surface, an exterior periphery, a first insulating material, and a passive resonance circuit having an inductor, a capacitor, and a resistor, wherein:
(a) said first insulating material is disposed on said implantable conductive stent, wherein said first insulating material is biocompatible, has a relative dielectric constant of from about 1.5 to about 10, and forms a liquid around said inductor; and
(b) the resonance frequency of said implantable stent assembly disposed in a human body corresponds substantially to a resonance frequency of a rotational frequency of the B1 field of the MR scanner applied by the magnetic resonance imaging system.

89. The implantable stent assembly as recited in claim 88, wherein said first insulating material is disposed on said inner surface of said implantable conductive stent.

90. The implantable stent assembly as recited in claim 88, wherein said first insulating material is disposed on said outer surface of said implantable conductive stent.

91. The implantable stent assembly as recited in claim 88, wherein said first insulating material has a relative dielectric constant from about two to about four.

92. The implantable stent assembly as recited in claim 88, wherein said first insulating material is a drug-eluting material.

93. An implantable stent assembly disposed in a human body and comprised of an implantable stent, a first electrically insulated wire, a second electrically insulated wire, a first insulating material, a first passive circuit having a first inductor, a first capacitor and a first resistor, a second passive circuit having a second inductor, a second capacitor and a second resistor, wherein said implantable stent assembly has a first resonance frequency and second resonance frequency when disposed in a human body, and wherein
(a) said implantable stent structure comprises a first strut, a second strut, a first end, a second end, an outer surface, at least two connector points, and at least four connector tabs;
(b) said first and second electrically insulated wires are disposed around said outer surface of said stent structure;
(c) said first and second electrically insulated wires are disposed from said first end of said stent structure to said second end of said stent structure and bend around to return to their starting points at said first end of said stent structure, thereby forming two return wires;
(d) said first end and second end of said first electrically insulated wire are electrically connected to said first capacitor;
(e) said first end and second end of said second electrically insulated wire are electrically connected to said second capacitor;
(f) said first and second capacitors are disposed on stent structure between said first stent strut and said second stent strut;
(g) said first and second capacitors further comprise a first conductive material, a second conductive material, a dielectric material and a second insulating material;
(h) said second insulating material is disposed on a portion of said outer surface of said stent structure between said first stent strut and said second stent strut;
(i) said first conductive material disposed over at least a portion of said second insulating material; a dielectric material disposed over at least a portion of said first conductive material; and a second conductive material disposed over at least a portion of said dielectric material;
(j) said first resonance frequency of said implantable stent assembly disposed in a human body corresponds substantially to a resonance frequency of a rotational frequency of the B1 field of the MR scanner applied by the magnetic resonance imaging system; and
(k) at least one of said first resonance frequency and said second resonance frequency of said implantable stent assembly disposed in a human body is within one kilohertz of the operating frequency of a magnetic resonance imaging system.

94. The implantable stent assembly as recited in claim 93, wherein said first conductive material is identical to said second conductive material.

95. The implantable stent assembly as recited in claim 93, wherein said first conductive material is different from said second conductive material.

96. The implantable stent assembly as recited in claim 93, wherein said electrical connection of said first insulated wire and said first capacitor comprises solder.

97. The implantable stent assembly as recited in claim 93, wherein said electrical connection of said second insulated wire and said second capacitor comprises solder.

98. The implantable stent assembly as recited in claim 93, wherein said electrical connection of said first insulated wire and said first capacitor comprises conductive epoxy.

99. The implantable stent assembly as recited in claim 93, wherein said electrical connection of said second insulated wire and said second capacitor comprises conductive epoxy.

100. The implantable stent assembly as recited in claim 93, wherein said first electrically insulated wire is disposed around said outer surface of said stent structure in a substantially coil shape with at least one inductor coil loop.

101. The implantable stent assembly as recited in claim 93, wherein said second electrically insulated wire is disposed around said outer surface of said stent structure in a substantially coil shape with at least one inductor coil loop.

102. The implantable stent assembly as recited in claim 93, wherein said second insulated wire is wound around said stent structure and extends from said first end of said stent structure to said second end of said stent structure to form an inductive coil at an orientation of about ninety degrees from said first electrically insulated wire, wherein said first and second ends of said second insulated wire are electrically connected to a single terminal capacitor on said stent structure at a connector point.

103. The implantable stent assembly as recited in claim 93, wherein
(a) said first passive circuit causes said implantable stent assembly to resonate at a frequency f1 when said implantable stent assembly is disposed in a human body; and
(b) said second passive circuit causes said implantable stent assembly to resonate at a frequency f2 when said implantable stent assembly is disposed in a human body.

104. The implantable stent assembly as recited in claim 103, wherein said frequency f2 is two times said frequency f1.

105. The implantable stent assembly as recited in claim 103, wherein said frequency f2 is substantially equal to said frequency f1.

106. The implantable stent assembly as recited in claim 103, wherein said frequency f2 and said frequency f1 are non-harmonic frequencies.

107. The implantable stent assembly as recited in claim 103, wherein said frequency f2 and said frequency f1 are harmonic frequencies.

108. The implantable stent assembly as recited in claim 103, wherein said frequency f1 is a frequency that corresponds substantially to a resonance frequency of a rotational frequency of the B1 field of the MR scanner applied by the magnetic resonance imaging system.

109. The implantable stent assembly as recited in claim 103, wherein said frequency f2 is a frequency that corresponds substantially to a resonance frequency of a rotational frequency of the B1 field of the MR scanner applied by the magnetic resonance imaging system.

110. The implantable stent assembly as recited in claim 103, wherein said frequency f1 is a frequency that corresponds substantially to a harmonic frequency of said resonance frequency of a rotational frequency of the B1 field of the MR scanner applied by the magnetic resonance imaging system.

111. The implantable stent assembly as recited in claim 103, wherein said frequency f2 is a frequency that corresponds substantially to a harmonic frequency of said resonance frequency of a rotational frequency of the B1 field of the MR scanner applied by the magnetic resonance imaging system.

112. The implantable stent assembly as recited in claim 93, wherein said first insulating material has a relative dielectric constant of from about 2 to about 4.

113. The implantable stent assembly as recited in claim 93, wherein said first insulating material has a resistivity of at least about 1×10¹² ohm-centimeters.

114. The implantable stent assembly as recited in claim 93, wherein said first insulating material is parylene.

115. The implantable stent assembly as recited in claim 93, wherein said first insulating material is aluminum nitride.

116. The implantable stent assembly as recited in claim 93, wherein said first insulating material is a polymeric material.

117. The implantable stent assembly as recited in claim 93, wherein said first conductive material has a resistivity of less than about 1.8×10⁻⁷ ohm-meters.

118. The implantable stent assembly as recited in claim 93, wherein said first conductive material is selected from the group consisting of copper, silver and gold.

119. The implantable stent assembly as recited in claim 93, wherein said dielectric material has a relative dielectric constant from about 1 to about 300.

120. The implantable stent assembly as recited in claim 93, wherein said dielectric material has a relative dielectric constant from about 1.5 to about 10.

121. The implantable stent assembly as recited in claim 93, wherein said dielectric material has a relative dielectric constant from about 2 to about 4.

122. The implantable stent assembly as recited in claim 93, wherein said second conductive material has a resistivity of less than about 1.8×10⁻⁷ ohm-meters.

123. The implantable stent assembly as recited in claim 93, wherein said second conductive material is selected from the group consisting of copper, silver and gold.

124. An implantable stent assembly disposed in a human body and comprised of an implantable stent, a first electrically insulated wire, a second electrically insulated wire, a first insulating material, a first passive circuit having a first inductor, a first capacitor and a first resistor, a second passive circuit having a second inductor, a second capacitor and a second resistor, wherein said implantable stent assembly has a first and second resonance frequencies when disposed in a human body, and wherein
(a) said implantable stent structure comprises a first strut, a second strut, a first end, a second end, an outer surface, at least two connector points, and at least four connector tabs;
(b) said first and second electrically insulated wires are disposed around said outer surface of said stent structure;
(c) said first and second electrically insulated wires are disposed from said first end of said stent structure to said second end of said stent structure and bend around to return to their starting points at said first end of said stent structure, thereby forming two return wires;
(d) said first end and second end of said first electrically insulated wire are electrically connected to said first capacitor;
(e) said first end and second end of said second electrically insulated wire are electrically connected to said second capacitor;
(f) said first and second capacitors are disposed on stent structure between said first stent strut and said second stent strut;
(g) said first and second capacitors further comprise a first conductive material, a second conductive material, a dielectric material and a second insulating material;
(h) said second insulating material is disposed on a portion of said outer surface of said stent structure between said first stent strut and said second stent strut;
(i) said first conductive material disposed over at least a portion of said second insulating material; a dielectric material disposed over at least a portion of said first conductive material; and a second conductive material disposed over at least a portion of said dielectric material; and
(j) and at least one of said first resonance frequency and said second resonance frequency of said implantable stent assembly disposed in a human body is in the range of from about one kilohertz above to about one kilohertz below the operating frequency of a magnetic resonance imaging system.

125. The implantable stent assembly as recited in claim 93, wherein said first resistor is disposed in series with said first capacitor and said first inductor.

126. An implantable stent assembly comprised of an implantable conductive stent with a longitudinal axis, an exterior periphery, a first end, a second end, at least one passive circuit having an inductor, a capacitor and a resistor, an electrically insulated wire, and an insulating material interposed between at least a portion of said implantable conductive stent and a portion of said passive circuit wherein
(a) said electrically insulated wire forms said inductor;
(b) said electrically insulated wire is disposed along the longitudinal axis from said first end of said implantable conductive stent to said second end of said implantable conductive stent forming a coil with a first end at its starting point, a second end at its ending point and at least one loop around the exterior periphery of said implantable conductive stent; and
(c) said electrically insulated wire further forms a return wire by traversing the longitudinal axis of said implantable conductive stent in a substantially straight line from said second end of said coil to said first end of said coil.

127. The implantable stent assembly as recited in claim 126, wherein said return wire is disposed over said coil.

128. The implantable stent assembly as recited in claim 126, wherein said return wire is disposed under said coil.

129. The implantable stent assembly as recited in claim 126, wherein said return wire is disposed and woven alternately over and under said coil.

130. An implantable stent assembly comprised of an implantable conductive stent with a longitudinal axis, a first end, a second end, at least two stent struts, at least one passive circuit having an inductor, a capacitor and a resistor, an electrically insulated wire, and an insulating material interposed between at least a portion of said implantable conductive stent and a portion of said passive circuit wherein
(a) said electrically insulated wire forms said inductor;
(b) said electrically insulated wire is disposed along the longitudinal axis from said first end of said implantable conductive stent to said second end of said implantable conductive stent forming a coil with at least one loop around said implantable conductive stent; and
(c) said electrically insulated wire is woven alternately over and under said stent struts.

131. An implantable stent assembly disposed in a human body and comprised of an implantable stent structure, a first insulating material, an electrically insulated wire comprising a first end, and at least one passive circuit having an inductor, a capacitor and a resistor, wherein said implantable stent assembly disposed in a human body has at least one resonance frequency, and wherein:
(a) said implantable stent structure comprises a first surface on which at least one electronic component is disposed, a first stent strut and a second stent strut; and
(b) said first surface comprises at least a portion of said first stent strut.

132. The implantable stent assembly as recited in claim 131, wherein
(a) said first stent strut and said second stent strut have a point of contact; and
(b) said first surface comprises said point of contact.

133. The implantable stent assembly as recited in claim 131, wherein said capacitor is disposed on said first surface.

134. The implantable stent assembly as recited in claim 130, wherein said capacitor comprises layered materials electrically connected to said first surface, and said first end of said electrically insulated wire is electrically connected to said electrical tab, thereby forming an RLC circuit.

135. An implantable stent assembly disposed in a human body and comprised of an implantable stent, a first electrically insulated wire, a second electrically insulated wire, a first insulating material, a first passive circuit having a first inductor, a first capacitor and a first resistor, a second passive circuit having a second inductor, a second capacitor and a second resistor, wherein said implantable stent assembly has a first and second resonance frequencies when disposed in a human body, and wherein
(a) said implantable stent structure comprises a first strut, a second strut, a first end, a second end, an outer surface, at least two connector points, and at least four connector tabs;
(b) said first and second electrically insulated wires are disposed around said outer surface of said stent structure;
(c) said first and second electrically insulated wires are disposed from said first end of said stent structure to said second end of said stent structure and bend around to return to their starting points at said first end of said stent structure, thereby forming two return wires;
(d) said first end and second end of said first electrically insulated wire are electrically connected to said first capacitor;
(e) said first end and second end of said second electrically insulated wire are electrically connected to said second capacitor;
(f) said first and second capacitors are disposed on stent structure between said first stent strut and said second stent strut;
(g) said first and second capacitors further comprise a first conductive material, a second conductive material, a dielectric material and a second insulating material;
(h) said second insulating material is disposed on a portion of said outer surface of said stent structure between said first stent strut and said second stent strut;
(i) said first conductive material disposed over at least a portion of said second insulating material; a dielectric material disposed over at least a portion of said first conductive material; and a second conductive material disposed over at least a portion of said dielectric material; and
(j) and at least one of said first resonance frequency and said second resonance frequency of said implantable stent assembly disposed in a human body is more than one kilohertz above or below the operating frequency of a magnetic resonance imaging system.

136. The implantable stent assembly as recited in claim 135, wherein said first conductive material is identical to said second conductive material.

137. The implantable stent assembly as recited in claim 135, wherein said first conductive material is different from said second conductive material.

138. The implantable stent assembly as recited in claim 135, wherein said electrical connection of said first insulated wire and said first capacitor comprises solder.

139. The implantable stent assembly as recited in claim 135, wherein said electrical connection of said second insulated wire and said second capacitor comprises solder.

140. The implantable stent assembly as recited in claim 135, wherein said electrical connection of said first insulated wire and said first capacitor comprises conductive epoxy.

141. The implantable stent assembly as recited in claim 135, wherein said electrical connection of said second insulated wire and said second capacitor comprises conductive epoxy.

142. The implantable stent assembly as recited in claim 135, wherein said first electrically insulated wire is disposed around said outer surface of said stent structure in a substantially coil shape with at least one inductor coil loop.

143. The implantable stent assembly as recited in claim 135, wherein said second electrically insulated wire is disposed around said outer surface of said stent structure in a substantially coil shape with at least one inductor coil loop.

144. The implantable stent assembly as recited in claim 135, wherein said second insulated wire is wound around said stent structure and extends from said first end of said stent structure to said second end of said stent structure to form an inductive coil at an orientation of about ninety degrees from said first electrically insulated wire, wherein said first and second ends of said second insulated wire are electrically connected to a single terminal capacitor on said stent structure at a connector point.

145. The implantable stent assembly as recited in claim 135, wherein
(a) said first passive circuit causes said implantable stent assembly to resonate at a frequency f1 when said implantable stent assembly is disposed in a human body; and
(b) said second passive circuit causes said implantable stent assembly to resonate at a frequency f2 when said implantable stent assembly is disposed in a human body.

146. The implantable stent assembly as recited in claim 145, wherein said frequency f2 is two times said frequency f1.

147. The implantable stent assembly as recited in claim 145, wherein said frequency f2 is substantially equal to said frequency f1.

148. The implantable stent assembly as recited in claim 145, wherein said frequency f2 and said frequency f1 are non-harmonic frequencies.

149. The implantable stent assembly as recited in claim 145, wherein said frequency f2 and said frequency f1 are harmonic frequencies.

150. The implantable stent assembly as recited in claim 145, wherein said frequency f1 is a frequency that corresponds substantially to a resonance frequency of a rotational frequency of the B1 field of the MR scanner applied by the magnetic resonance imaging system.

151. The implantable stent assembly as recited in claim 145, wherein said frequency f2 is a frequency that corresponds substantially to a resonance frequency of a rotational frequency of the B1 field of the MR scanner applied by the magnetic resonance imaging system.

152. The implantable stent assembly as recited in claim 145, wherein said frequency f1 is a frequency that corresponds substantially to a harmonic frequency of said resonance frequency of a rotational frequency of the B1 field of the MR scanner applied by the magnetic resonance imaging system.

153. The implantable stent assembly as recited in claim 145, wherein said frequency f2 is a frequency that corresponds substantially to a harmonic frequency of said resonance frequency of a rotational frequency of the B1 field of the MR scanner applied by the magnetic resonance imaging system.

154. The implantable stent assembly as recited in claim 135, wherein said first insulating material has a relative dielectric constant of from about 2 to about 4.

155. The implantable stent assembly as recited in claim 135, wherein said first insulating material has a resistivity of at least about 1×10¹² ohm-centimeters.

156. The implantable stent assembly as recited in claim 135, wherein said first insulating material is parylene.

157. The implantable stent assembly as recited in claim 135, wherein said first insulating material is aluminum nitride.

158. The implantable stent assembly as recited in claim 135, wherein said first insulating material is a polymeric material.

159. The implantable stent assembly as recited in claim 135, wherein said first conductive material has a resistivity of less than about 1.8×10⁻⁷ ohm-meters.

160. The implantable stent assembly as recited in claim 135, wherein said first conductive material is selected from the group consisting of copper, silver and gold.

161. The implantable stent assembly as recited in claim 135, wherein said dielectric material has a relative dielectric constant from about 1 to about 300.

162. The implantable stent assembly as recited in claim 135, wherein said dielectric material has a relative dielectric constant from about 1.5 to about 10.

163. The implantable stent assembly as recited in claim 135, wherein said dielectric material has a relative dielectric constant from about 2 to about 4.

164. The implantable stent assembly as recited in claim 135, wherein said second conductive material has a resistivity of less than about 1.8×10⁻⁷ ohm-meters.

165. The implantable stent assembly as recited in claim 135, wherein said second conductive material is selected from the group consisting of copper, silver and gold.

166. The implantable stent assembly as recited in claim 135, wherein said first resistor is disposed in series with said first capacitor and said first inductor.
